(19) United States
(12) Patent Application Publication
(10) Pub. No.: US 2013/0203815 A1
(43) Pub. Date: Aug. 8, 2013

(54) CYTOKINE INHIBITORS

(71) Applicants: Babasaheb Pandurang Bandgar, Solapur (IN); Jalindar Vasant Totre, Pune (IN)

(72) Inventors: Babasaheb Pandurang Bandgar, Solapur (IN); Jalindar Vasant Totre, Pune (IN)

(73) Assignee: PIRAMAL ENTERPRISES LIMITED, Mumbai (IN)

(21) Appl. No.: 13/637,755

(22) PCT Filed: Mar. 25, 2011
(86) PCT No.: PCT/IB11/51269

§ 371 Date: Nov. 8, 2012

Related U.S. Application Data
(60) Provisional application No. 61/318,470, filed on Mar. 29, 2010.

(51) Int. Cl. C07D 409/10; C07D 211/42; C07D 405/10; C07D 207/08; C07D 257/04; C07D 211/70; C07D 207/16

(52) U.S. Cl. CPC C07D 409/10; C07D 211/70; C07D 211/42; C07D 207/16; C07D 207/08; C07D 257/04; C07D 405/10
USPC 514/327; 546/339; 514/277; 546/216; 548/572; 514/428; 548/570; 548/571; 548/253; 514/381; 548/517; 514/422; 548/527

(57) ABSTRACT

The present invention provides compounds represented by general formula (I):

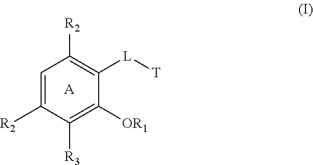

wherein, R₁, R₂, R₃, L and T are as defined in the specification, in all their stereoisomeric and tautomeric forms and mixtures thereof in all ratios, and their pharmaceutically acceptable salts, pharmaceutically acceptable solvates, and prodrugs thereof. The invention also relates to processes for the manufacture of compounds of formula (I) and pharmaceutical compositions containing them. The compounds and the pharmaceutical compositions of the present invention are useful in the treatment of a condition or disorder mediated by one or more cytokines selected from Tumor Necrosis Factor-alpha (TNF-α) and interleukins such as IL-1, IL-6, and IL-8. The present invention further provides a method of treatment of inflammatory disorders by administering a therapeutically effective amount of the said compound of formula (I) or its pharmaceutical composition, to a mammal in need thereof.

### FIELD OF THE INVENTION

The present invention relates to phenyl derivatives, processes for their preparation, pharmaceutical compositions containing them, and use of these compounds and pharmaceutical compositions containing them for the treatment of a condition or a disorder mediated by one or more cytokines selected from Tumor Necrosis Factor-alpha (TNF-α) and interleukins such as IL-1, IL-6 or IL-8.

### BACKGROUND OF THE INVENTION

Cytokines, especially TNF-α, IL-1β, IL-6, and IL-8 play an important role in the inflammatory process.

Tumor Necrosis Factor-α (TNF-α) is a soluble homotrimer of 17 kD protein subunits. Monocytes and macrophages secrete cytokines such as TNF-α, interleukin-1 (IL-1) and interleukin-6 (IL-6) in response to endotoxin or other stimuli. TNF-α is also produced by cells other than monocytes or macrophages. TNF-α demonstrates beneficial as well as pathological activities. TNF-α has been implicated in inflammatory diseases, autoimmune diseases, viral, bacterial and parasitic infections, malignancies, and/or neurogenerative diseases, and is a useful target for specific biological therapy in diseases such as rheumatoid arthritis and Crohn's disease.

Interleukin-1 (IL-1) is an important part of the innate immune system, which regulates functions of the adaptive immune system. The balance between IL-1 and IL-1 receptor antagonist (IL-1 ra) in local tissues influences the possible development of an inflammatory disease and resultant structural damage. In the presence of an excess amount of IL-1, inflammatory and autoimmune disorders may be developed in joints, lungs, gastrointestinal tract, central nervous system (CNS) or blood vessels.

Interleukin-6 (IL-6) is a polypeptide cytokine consisting of 184 amino acids with a molecular weight of 21 to 28 kDa. IL-6 is produced from a wide variety of cells such as vascular endothelial cells, T-lymphocytes, B-lymphocytes, monocytes, and macrophages by various kinds of stimulative substances such as lipopolysaccharide, IL-1, and TNF, which can be found at the site of inflammation.

Inflammation is the response of a tissue to injury that may be caused by invading parasites, ischemia, antigen-antibody reactions or other forms of physical or chemical injury. It is characterized by increased blood flow to the tissue, causing pyrexia, redness, swelling, and pain.

Both TNF-α and/or interleukins (IL-1, IL-6, IL-8) induce the expression of a variety of genes that contribute to the inflammatory process. An increase in TNF-α synthesis/release is a common phenomenon during the inflammatory process. Inflammation is an inherent part of various disease states like rheumatoid arthritis, Crohn's disease, septic shock syndrome, atherosclerosis, among other clinical conditions.

Among other inflammatory diseases, Rheumatoid arthritis (RA)—an autoimmune disorder, is a chronic, systemic, articular inflammatory disease of unknown etiology. In RA, the normally thin synovial lining of joints is replaced by an inflammatory, highly vascularized, invasive fibrocollagenase tissue (pannus), which is destructive to both cartilage and bone. Areas that may be affected include the joints of the hands, wrists, neck, jaw, elbows, feet and ankles. Cartilage destruction in RA is linked to aberrant cytokines and growth factor expression in the affected joints.

The most common rheumatoid arthritis therapy involves the use of nonsteroidal anti-inflammatory drugs (NSAIDs) to alleviate symptoms. However, despite the widespread use of NSAIDs, many individuals cannot tolerate the doses necessary to treat the disorder over a prolonged period of time. In addition, NSAIDs merely treat the symptoms of disorder and not the cause.

When patients fail to respond to NSAIDs, other drugs such as methotrexate, gold salts, D-penicillamine and prednisone are used. These drugs also have significant toxicities and their mechanism of action remains unknown.

There are several small molecules which inhibit the production of inflammatory cytokines and have demonstrated activity in animal rheumatoid arthritis models. Such molecules are in various stages of preclinical and clinical development (Nature Reviews, 2003, 2, 736-746).

U.S. Pat. No. 5,589,514, U.S. Pat. No. 5,776,977 and U.S. Pat. No. 6,159,988 describe arylcycloalkyl derivatives useful in the treatment of inflammatory conditions.

The present inventors have synthesized phenyl derivatives which are inhibitors of one or more cytokines selected from TNF-α, IL-1, IL-6, or IL-8 and are useful for the treatment of inflammatory disorders.

### SUMMARY OF THE INVENTION

Thus according to one aspect of the present invention there are provided compounds of formula (I) (as described herein below), as well as stereoisomers, tautomeric forms, pharmaceutically acceptable salts, solvates and prodrugs thereof.

According to another aspect of the present invention, there are provided compounds of formula (I), which are inhibitors of one or more cytokines selected from TNF-α, IL-1, IL-6 or IL-8.

According to another aspect of the present invention, there are provided processes for producing compounds of formula (I).

According to another aspect of the invention, there are provided pharmaceutical compositions comprising one or more compounds of formula (I) as active ingredients useful in the treatment of a condition or disorder mediated by one or more cytokines selected from TNF-α, IL-1, IL-6 or IL-8.

According to another aspect of the present invention there are provided methods for the manufacture of medicaments comprising compounds of formula (I), which are useful for the treatment of a condition or disorder mediated by one or more cytokines selected from TNF-α, IL-1, IL-6 or IL-8.

According to another aspect of the present invention there is provided a method for the treatment of conditions or disorders mediated by one or more cytokines selected from TNF-α, IL-1, IL-6 or IL-8, administering to a mammal in need thereof a therapeutically effective amount of the compound of formula (I).

According to a further aspect of the present invention, there is provided use of compounds of formula (I) for the treatment of a condition or disorder mediated by one or more cytokines selected from TNF-α, IL-1, IL-6 or IL-8.

These and other objectives and advantages of the present invention will be apparent to those skilled in the art from the following description.

### DETAILED DESCRIPTION OF THE INVENTION

The present invention provides compounds of formula (I), in all their stereoisomeric and tautomeric forms and mixtures thereof in all ratios, their pharmaceutically acceptable salts, pharmaceutically acceptable solvates, and prodrugs thereof;

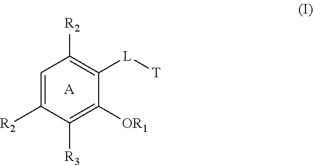

wherein,
R₁ is selected from hydrogen, alkyl or —C(O)-alkyl;
R₂ at each occurrence is independently selected from hydrogen, halogen, hydroxy, alkyl, alkoxy or —O—C(O)-alkyl;
R₃ is selected from the groups of formula (i) to (iv)

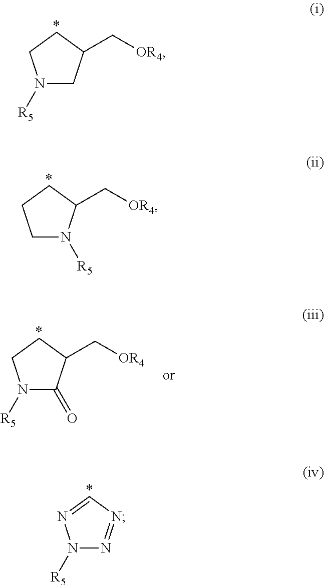

* indicates the point of attachment;
R₄ is selected from hydrogen, alkyl or —C(O)-alkyl;
R₅ is selected from hydrogen or alkyl;
L is selected from the groups of formula:

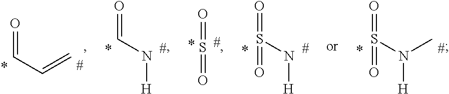

* indicates the point of attachment to phenyl ring A;
# indicates the point of attachment to T; and
T is selected from phenyl or 5 or 6 membered heteroaryl; wherein the phenyl and heteroaryl are unsubstituted or substituted by at least one group selected from halogen, hydroxy, alkyl, haloalkyl, alkoxy, carboxy, amino, nitro or cyano.

### DEFINITIONS

Listed below are definitions, which apply to the terms as they are used throughout the specification and the appended claims (unless they are otherwise limited in specific instances), either individually or as part of a larger group.

It will be understood that “substitution” or “substituted with” includes the implicit proviso that such substitution is in accordance with permitted valence of the substituted atom and the substituent, and results in a stable compound, which does not readily undergo transformation such as by rearrangement, cyclization, elimination, etc.

As used herein, the term “alkyl” refers to a saturated aliphatic group, including straight or branched-chain alkyl group containing 1-10 carbon atoms. Suitable examples of alkyl groups containing from 1 to 6 carbon atoms include methyl, ethyl, propyl, butyl, pentyl, hexyl, isopropyl, isobutyl, 1-methylbutyl, isopentyl, neopentyl, 2,2-dimethylbutyl, 2-methylpentyl, 3-methylpentyl, isohexyl, sec-butyl, and tert-butyl. The “alkyl” may optionally be substituted by one or more substituents selected from halogen, hydroxy, carboxy, acetoxy, amino, cycloalkyl, haloalkyl, alkoxy, aryloxy, alkoxycarbonyl, aminocarbonyl, aminoaryl, aryl, and heterocyclyl.

The term “alkoxy” unless otherwise stated, denotes alkyl group as defined above attached via oxygen linkage to the rest of the molecule. Representative examples of alkoxy groups include methoxy, ethoxy, and propoxy.

The term “cycloalkyl” refers to a saturated mono-, or bi-cyclic ring system containing a specified number of carbon atoms. Cycloalkyls have 3, 4, 5, 6 or 7 carbon atoms in each ring structure. Examples of cycloalkyl residues containing 3, 4, 5, 6 or 7 ring carbon atoms are cyclopropyl, cyclobutyl, cyclopentyl, cyclohexyl, and cycloheptyl.

As used herein, the term “aryl” refers to a monocyclic or polycyclic hydrocarbon group having up to 10 ring carbon atoms, in which at least one carbocyclic ring is present that has a conjugated π electron system. Examples of aryl residues include phenyl, and napthyl. The “aryl” is optionally substituted by one or more substituents selected from halogen, hydroxy, alkoxy, oxo, alkyl, haloalkyl, heterocyclyl, amino, nitro, cyano, aryl, and carboxy.

The term “heteroatom” refers to nitrogen, oxygen and sulfur. It should be noted that any heteroatom with unsatisfied valences is assumed to have a hydrogen atom to satisfy the valences. The ring heteroatoms can be present in any desired number and in any position with respect to each other provided that the resulting heterocyclic system is stable and suitable as a subgroup in a drug substance.

The term “heterocyclyl” refers to a saturated or unsaturated monocyclic ring system containing 5 or 6, ring atoms of which 1 or 2 are identical or different heteroatoms selected from: nitrogen, oxygen and sulfur. Suitable examples of such heterocyclyl groups are pyrrolyl, imidazolyl, pyrrolidinyl, pyridinyl, pyrazinyl, pyridazinyl, pyrimidinyl, pyrazolyl, piperidinyl, piperazinyl, morpholinyl, and thiomorpholinyl. The “heterocyclyl” is optionally substituted by one or more substituents selected from halogen, hydroxy, alkoxy, oxo, alkyl, haloalkyl, heterocyclyl, amino, nitro, cyano, aryl, and carboxy.

The term “heteroaryl” as used herein refers to an unsaturated monocyclic heterocyclic ring system containing 5 or 6 ring atoms, The rings may contain from one to four hetero atoms selected from N, O or S, wherein the N or S atom(s) are optionally oxidized, or the N atom(s) are optionally quaternized. Any suitable ring position of the heteroaryl moiety may be covalently linked to the defined chemical structure. Examples of heteroaryl include furan, thiophene, pyrrole, pyrazole, imidazole, oxazole, isoxazole, thiazole, isothiazole, 1H-tetrazole, oxadiazole, triazole, pyridine, pyrimidine, pyrazine, and pyridazine. The “heteroaryl” is optionally substituted by one or more substituents selected from halogen, hydroxy, alkoxy, oxo, alkyl, haloalkyl, heterocyclyl, amino, nitro, cyano, aryl, and carboxy.

The term “halogen” or “halo” unless otherwise stated refer to fluorine, chlorine, bromine, or iodine atom.

The term “amino” refers to the group —NH₂ which may be optionally substituted by one or more substituents selected from alkyl or aryl.

The term “pharmaceutically acceptable” as used herein means that the carrier, diluent, excipients, and/or salt must be compatible with the other ingredients of the formulation, and not deleterious to the recipient thereof.

The term “mammal” used herein refers to warm-blooded vertebrate animals of the class Mammalia, including humans, characterized by a covering of hair on the skin and, in the female, milk-producing mammary glands for nourishing the young. The term mammal includes animals such as cat, dog, rabbit, bear, fox, wolf, monkey, deer, mouse, pig as well as human.

As used herein, the terms “treat” or, “therapy” refer to alleviate, or slow the progression, prophylaxis, attenuation or cure of existing disease, condition or disorder.

The term “inflammatory disorder” as used herein refers to a disease, disorder or a condition characterized by chronic inflammation including rheumatoid arthritis, osteoarthritis, juvenile rheumatoid arthritis, psoriatic arthritis, refractory rheumatoid arthritis, chronic non-rheumatoid arthritis, ankylosing spondylitis, Behcet's disease, osteoporosis/bone resorption, coronary heart disease, atherosclerosis, vasculitis, ulcerative colitis, psoriasis, Crohn's disease, adult respiratory distress syndrome, delayed-type hypersensitivity in skin disorders, septic shock syndrome, and inflammatory bowel disease.

As used herein the term “prodrug” refers to compounds that are drug precursors, which following administration into or onto the body, release the drug in vivo via a chemical or physiological process e.g., a prodrug on being brought to the physiological pH or through an enzyme action is converted to the desired drug form.

In an embodiment, the present invention provides compounds of formula (I), in all their stereoisomeric and tautomeric forms and mixtures thereof in all ratios, their pharmaceutically acceptable salts, pharmaceutically acceptable solvates, and prodrugs thereof;

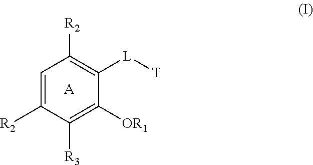

wherein,
R₁ is selected from hydrogen or alkyl;
R₂ at each occurrence is independently selected from hydrogen, halogen, hydroxy, alkyl or alkoxy;
R₃ is selected from the groups of formula (i) to (iv)

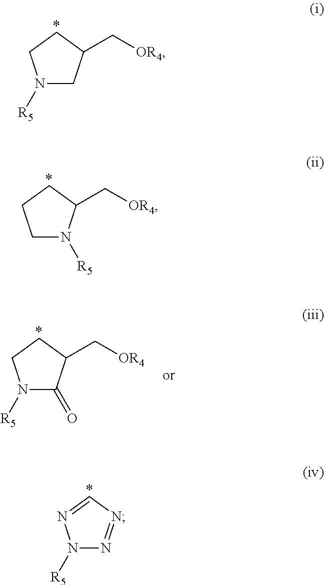

* indicates the point of attachment;
R₄ is selected from hydrogen, alkyl or —C(O)-alkyl;
R₅ is selected from hydrogen or alkyl;
L is selected from the groups of formula:

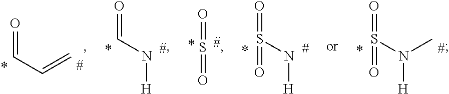

* indicates the point of attachment to phenyl ring A;
# indicates the point of attachment to T; and
T is selected from phenyl or 5 or 6 membered heteroaryl; wherein the phenyl and heteroaryl are unsubstituted or substituted by at least one group selected from halogen, hydroxy, alkyl, haloalkyl, alkoxy, carboxy, amino, nitro or cyano.

In an embodiment, the present invention provides compounds of formula (I), in all their stereoisomeric and tautomeric forms and mixtures thereof in all ratios, their pharmaceutically acceptable salts, pharmaceutically acceptable solvates, and prodrugs thereof; wherein,

R₁ is selected from hydrogen or alkyl;
R₂ at each occurrence is independently selected from hydrogen, halogen, hydroxy, alkyl or alkoxy;
R₃ is selected from the groups of formula (i) to (iv)

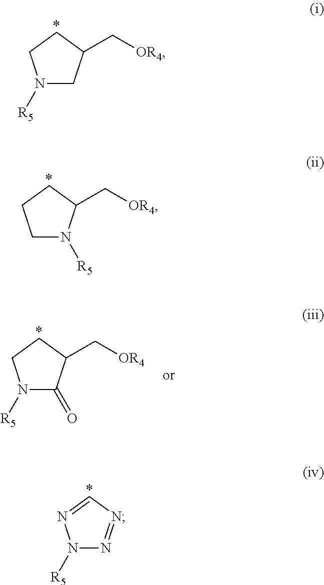

* indicates the point of attachment;

R₄ is selected from hydrogen, alkyl or —C(O)-alkyl;
R₅ is selected from hydrogen or alkyl;
L is selected from the groups of formula:

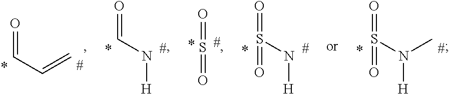

* indicates the point of attachment to phenyl ring A;
# indicates the point of attachment to T; and
T is phenyl; which is unsubstituted or substituted by at least one group selected from halogen, hydroxy, alkyl, haloalkyl, alkoxy, carboxy, amino, nitro or cyano.

In a further embodiment, the present invention provides compounds of formula (I), in all their stereoisomeric and tautomeric forms and mixtures thereof in all ratios, their pharmaceutically acceptable salts, pharmaceutically acceptable solvates, and prodrugs thereof; wherein,

R₁ is hydrogen;
R₂ is alkoxy;
R₃ is group of formula (ii)

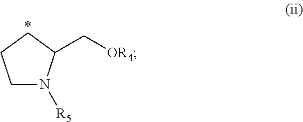

* indicates the point of attachment;

R₄ is selected from hydrogen or —C(O)-alkyl;
R₅ is alkyl;
L is selected from the groups of formula:

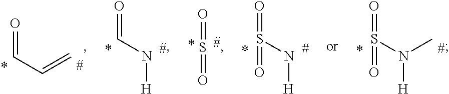

* indicates the point of attachment to phenyl ring A;
# indicates the point of attachment to T; and
T is phenyl; which is unsubstituted or substituted by at least one group selected from halogen, hydroxy, alkyl, haloalkyl, alkoxy, carboxy, amino, cyano or nitro.

In a further embodiment, the present invention provides compounds of formula (I), in all their stereoisomeric and tautomeric forms and mixtures thereof in all ratios, their pharmaceutically acceptable salts, pharmaceutically acceptable solvates, and prodrugs thereof; wherein,

R₁ is hydrogen;
R₂ is alkoxy;
R₃ is group of formula (ii)

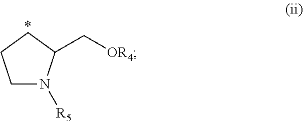

* indicates the point of attachment;

R₄ is selected from hydrogen or —C(O)-alkyl;
R₅ is alkyl;

### L is

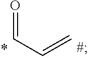

* indicates the point of attachment to phenyl ring A;
# indicates the point of attachment to T; and
T is phenyl; which is unsubstituted or substituted by at least one group selected from halogen, hydroxy, alkyl, haloalkyl, alkoxy, carboxy, amino, cyano or nitro.

In a further embodiment, the present invention provides compounds of formula (I), in all their stereoisomeric and tautomeric forms and mixtures thereof in all ratios, their pharmaceutically acceptable salts, pharmaceutically acceptable solvates, and prodrugs thereof; wherein,

R₁ is hydrogen;
R₂ is alkoxy;
R₃ is selected from the groups of formula (I), (iii) or (iv);

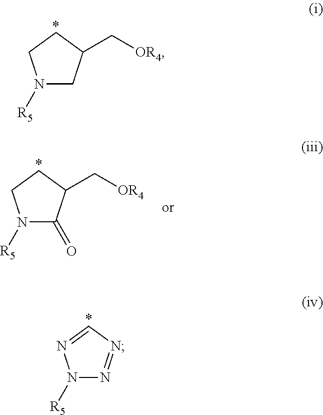

* indicates the point of attachment;

R₄ is hydrogen;
R₅ is alkyl;

### L is:

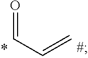

* indicates the point of attachment to phenyl ring A;
# indicates the point of attachment to T; and
T is phenyl; which is unsubstituted or substituted by at least one group selected from halogen, hydroxy, alkyl, haloalkyl, alkoxy, carboxy, amino, cyano or nitro.

In a further embodiment, the present invention provides compounds of formula (I), in all their stereoisomeric and tautomeric forms and mixtures thereof in all ratios, their pharmaceutically acceptable salts, pharmaceutically acceptable solvates, and prodrugs thereof; wherein,

R₁ is selected from hydrogen or alkyl;
R₂ at each occurrence is independently selected from hydrogen, halogen, hydroxy, alkyl or alkoxy;
R₃ is selected from the groups of formula (i) to (iv)

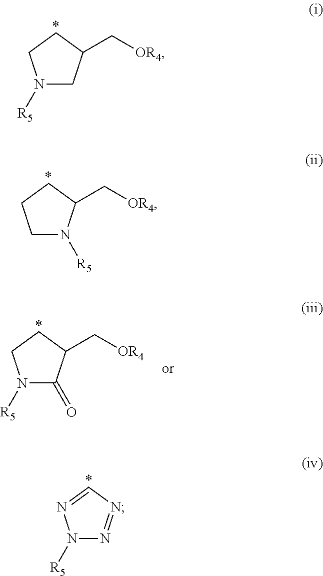

* indicates the point of attachment;

R₄ is selected from hydrogen, alkyl or —C(O)-alkyl;
R₅ is selected from hydrogen or alkyl;
L is selected from the groups of formula:

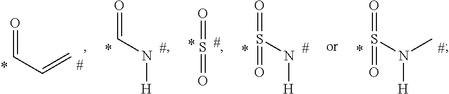

* indicates the point of attachment to phenyl ring A;
# indicates the point of attachment to T; and
T is 5 or 6 membered heteroaryl; wherein the heteroaryl is unsubstituted or substituted by at least one group selected from halogen, nitro, amino, alkoxy, carboxy, alkyl, haloalkyl, cyano or hydroxy.

In a further embodiment, the present invention provides compounds of formula (I), in all their stereoisomeric and tautomeric forms and mixtures thereof in all ratios, their pharmaceutically acceptable salts, pharmaceutically acceptable solvates, and prodrugs thereof; wherein,

R₁ is hydrogen;
R₂ is alkoxy;
R₃ is a group of formula (ii)

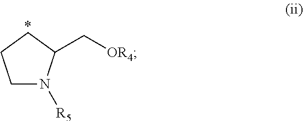

* indicates the point of attachment;
R₄ is hydrogen;
R₅ is alkyl;
L is selected from the groups of formula:

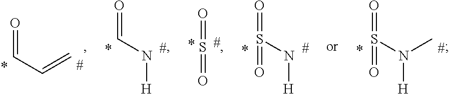

* indicates the point of attachment to phenyl ring A;
# indicates the point of attachment to T; and
T is 5 membered heteroaryl selected from of furanyl, thiophenyl, imidazolyl, oxazolyl, isoxazolyl or thiazolyl; wherein the heteroaryl is unsubstituted or substituted by at least one group selected from halogen, nitro, amino, alkoxy, carboxy, alkyl, haloalkyl, cyano or hydroxy.

In a further embodiment, the present invention provides compounds of formula (I), in all their stereoisomeric and tautomeric forms and mixtures thereof in all ratios, their pharmaceutically acceptable salts, pharmaceutically acceptable solvates, and prodrugs thereof; wherein,

R₁ is hydrogen;
R₂ is alkoxy;
R₃ is a group of formula (ii)

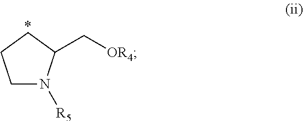

*indicates the point of attachment;

R₄ is hydrogen;
R₅ is alkyl;

### L is:

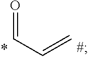

* indicates the point of attachment to phenyl ring A;
# indicates the point of attachment to T; and
T is 5 membered heteroaryl selected from furanyl, thiophenyl, imidazolyl, oxazolyl, isoxazolyl or thiazolyl; wherein the heteroaryl is unsubstituted or substituted by at least one group selected from halogen, nitro, amino, alkoxy, carboxy, alkyl, cyano or hydroxy.

In a further embodiment, the present invention provides compounds of formula (I), in all their stereoisomeric and tautomeric forms and mixtures thereof in all ratios, their pharmaceutically acceptable salts, pharmaceutically acceptable solvates, and prodrugs thereof; wherein,

R₁ is hydrogen;
R₂ is alkoxy;
R₃ is a group of formula (ii)

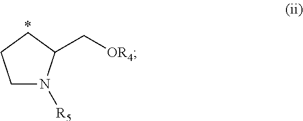

* indicates the point of attachment;

R₄ is hydrogen;
R₅ is alkyl;

### L is

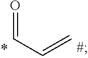

* indicates the point of attachment to phenyl ring A;
# indicates the point of attachment to T; and
T is selected from furanyl or thiophenyl; wherein the furanyl and thiophenyl are unsubstituted or substituted by at least one group selected from halogen or alkyl.

In a further embodiment, the present invention provides compounds of formula (I), in all their stereoisomeric and tautomeric forms and mixtures thereof in all ratios, their pharmaceutically acceptable salts, pharmaceutically acceptable solvates, and prodrugs thereof; wherein,

R₁ is hydrogen;
R₂ is alkoxy;
R₃ is a group of formula (ii)

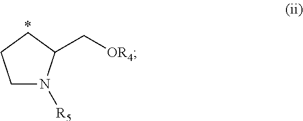

* indicates the point of attachment;

R₄ is hydrogen;
R₅ is alkyl;
L is selected from groups of formula:

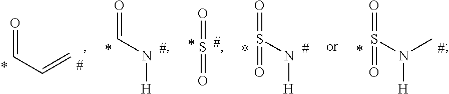

* indicates the point of attachment to phenyl ring A;
# indicates the point of attachment to T; and
T is 6 membered heteroaryl selected from pyrazinyl, pyridinyl, pyrimidinyl, or pyridazinyl; wherein the heteroaryl is unsubstituted or substituted by at least one group selected from halogen, nitro, amino, alkoxy, carboxy, alkyl, haloalkyl, cyano or hydroxy.

In a further embodiment, the present invention provides compounds of formula (I), in all their stereoisomeric and tautomeric forms and mixtures thereof in all ratios, their pharmaceutically acceptable salts, pharmaceutically acceptable solvates, and prodrugs thereof; wherein,

R₁ is hydrogen;
R₂ is alkoxy;
R₃ is a group of formula (ii)

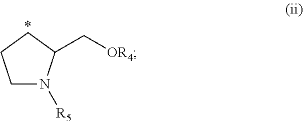

* indicates the point of attachment;
R₄ is hydrogen;
R₅ is alkyl;

### L is

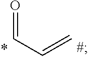

* indicates the point of attachment to phenyl ring A;
# indicates the point of attachment to T; and
T is 6 membered heteroaryl selected from pyrazinyl, pyridinyl, pyrimidinyl, or pyridazinyl; wherein the heteroaryl is unsubstituted or substituted by at least one group selected from halogen, nitro, amino, alkoxy, carboxy, alkyl, cyano or hydroxy.

In a further embodiment, the present invention provides compounds of formula (I), in all their stereoisomeric and tautomeric forms and mixtures thereof in all ratios, their pharmaceutically acceptable salts, pharmaceutically acceptable solvates, and prodrugs thereof; wherein,

R₁ is hydrogen;
R₂ is alkoxy;
R₃ is a group of formula (ii)

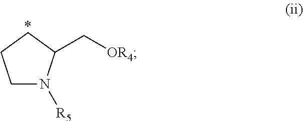

* indicates the point of attachment;
R₄ is hydrogen;
R₅ is alkyl;

### L is

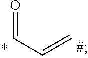

* indicates the point of attachment to phenyl ring A;
# indicates the point of attachment to T; and
T is pyridinyl; wherein the pyridinyl is unsubstituted or substituted by at least group selected from halogen or alkyl.

In a further embodiment, the present invention provides compounds of formula (I), in all their stereoisomeric and tautomeric forms and mixtures thereof in all ratios, their pharmaceutically acceptable salts, pharmaceutically acceptable solvates, and prodrugs thereof; wherein,

R₁ is hydrogen;
R₂ is alkoxy;
R₃ is selected from the groups of formula (i), (iii) or (iv);

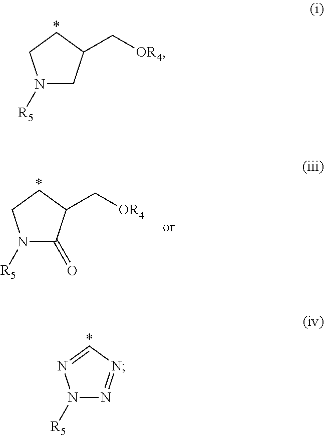

* indicates the point of attachment;

R₄ is hydrogen;
R₅ is alkyl;

### L is

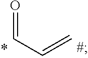

* indicates the point of attachment to phenyl ring A;
# indicates the point of attachment to T; and
T is 5 or 6 membered heteroaryl; wherein the heteroaryl is unsubstituted or substituted by at least one group selected from halogen, nitro, amino, alkoxy, carboxy, alkyl, haloalkyl, cyano or hydroxy.

Exemplary compounds of the present invention are selected from,
(+/−)3-(2-Chlorophenyl)-1-(2-hydroxy-3-(2-(hydroxymethyl)-1-methylpyrrolidin-3-yl)-4,6-dimethoxy-phenyl)prop-2-en-1-one hydrochloride,
(−)3-(2-Chlorophenyl)-1-(2-hydroxy-3-(2-(hydroxymethyl)-1-methylpyrrolidin-3-yl)-4,6-dimethoxy-phenyl)prop-2-en-1-one,
(+)3-(2-Chlorophenyl)-1-(2-hydroxy-3-(2-(hydroxymethyl)-1-methylpyrrolidin-3-yl)-4,6-dimethoxy-phenyl)prop-2-en-1-one,
(+/−)3-(3-Bromophenyl)-1-(2-hydroxy-3-(2-(hydroxymethyl)-1-methylpyrrolidin-3-yl)-4,6-dimethoxy-phenyl)prop-2-en-1-one,
(+/−)3-(2,4-Dimethoxy-phenyl)-1-(2-hydroxy-3-(2-(hydroxymethyl)-1-methyl pyrrolidin-3-yl)-4,6-dimethoxy-phenyl)prop-2-en-1-one,
(+/−)1-(2-Hydroxy-3-(2-(hydroxymethyl)-1-methylpyrrolidin-3-yl)-4,6-dimethoxy-phenyl)-3-m-tolylprop-2-en-1-one,
(+/−)1-(2-Hydroxy-3-(2-(hydroxymethyl)-1-methylpyrrolidin-3-yl)-4,6-dimethoxy-phenyl)-3-o-tolylprop-2-en-1-one,
(+/−)1-(2-Hydroxy-3-(2-(hydroxymethyl)-1-methylpyrrolidin-3-yl)-4,6-dimethoxy-phenyl)-3-(3-nitrophenyl)prop-2-en-1-one,
(+/−)1-(2-Hydroxy-3-(2-(hydroxymethyl)-1-methylpyrrolidin-3-yl)-4,6-dimethoxy-phenyl)-3-(4-nitrophenyl)prop-2-en-1-one,
(+/−)3-(2-Bromophenyl)-1-(2-hydroxy-3-(2-(hydroxymethyl)-1-methylpyrrolidin-3-yl)-4,6-dimethoxy-phenyl)prop-2-en-1-one,
(+/−)3-(4-Chlorophenyl)-1-(2-hydroxy-3-(2-(hydroxymethyl)-1-methylpyrrolidin-3-yl)-4,6-dimethoxy-phenyl)prop-2-en-1-one,
(+/−)3-(4-Fluorophenyl)-1-(2-hydroxy-3-(2-(hydroxymethyl)-1-methylpyrrolidin-3-yl)-4,6-dimethoxy-phenyl)prop-2-en-1-one,
(+/−)1-(2-Hydroxy-3-(2-(hydroxymethyl)-1-methylpyrrolidin-3-yl)-4,6-dimethoxy-phenyl)-3-(4-methoxyphenyl)prop-2-en-1-one,
(+/−)1-(2-Hydroxy-3-(2-(hydroxymethyl)-1-methylpyrrolidin-3-yl)-4,6-dimethoxy-phenyl)-3-p-tolylprop-2-en-1-one,
(+/−)1-(2-Hydroxy-3-(2-(hydroxymethyl)-1-methylpyrrolidin-3-yl)-4,6-dimethoxy-phenyl)-3-phenylprop-2-en-1-one,
(+/−)3-(3-Aminophenyl)-1-(2-hydroxy-3-(2-(hydroxymethyl)-1-methylpyrrolidin-3-yl)-4,6-dimethoxy-phenyl)prop-2-en-1-one,
(+/−) Acetic acid-(3-(3-(3-(2-chlorophenyl)acryloyl)-2-hydroxy-4,6-dimethoxy-phenyl)-1-methylpyrrolidin-2-ylmethyl ester,
(+/−)3-(3-Fluorophenyl)-1-(2-hydroxy-3-(2-(hydroxymethyl)-1-methylpyrrolidin-3-yl)-4,6-dimethoxy-phenyl)prop-2-en-1-one,
(+/−)3-(2-Fluorophenyl)-1-(2-hydroxy-3-(2-(hydroxymethyl)-1-methylpyrrolidin1-3-yl)-4,6-dimethoxy-phenyl)prop-2-en-1-one,
(+/−)1-(2-Hydroxy-3-(2-(hydroxymethyl)-1-methylpyrrolidin-3-yl)-4,6-dimethoxy-phenyl)-3-(3-hydroxy-4-methoxyphenyl)prop-2-en-1-one,
(+/−)3-(2-Chlorophenyl)-1-(2-hydroxy-3-(4-(hydroxymethyl)-1-methylpyrrolidin-3-yl)-4,6-dimethoxy-phenyl)prop-2-en-1-one,
(+/−)4-(3-(3-(2-Chlorophenyl)acryloyl)-2-hydroxy-4,6-dimethoxy-phenyl)-3-(hydroxymethyl)-1-methylpyrrolidin-2-one,
(+/−)3-(3-Bromophenyl)-1-(2-hydroxy-4,6-dimethoxy-3-(2-methyl-2H tetrazol-5-yl)phenyl)prop-2-en-1-one,
(+/−)1-(2-Hydroxy-3-(2-(hydroxymethyl)-1-methylpyrrolidin-3-yl)-4,6-dimethoxy-phenyl)-3-(4-methylfuran-2-yl)prop-2-en-1-one,
(+/−)3-(5-Bromofuran-2-yl)-1-(2-hydroxy-3-(2-(hydroxymethyl)-1-methylpyrrolidin-3-yl)-4,6-dimethoxy-phenyl)prop-2-en-1-one,
(+/−)3-(4-Bromothiophen-2-yl)-1-(2-hydroxy-3-(2-(hydroxymethyl)-1-methylpyrrolidin-3-yl)-4,6-dimethoxy-phenyl)prop-2-en-1-one,
(+/−)1-(2-Hydroxy-3-(2-(hydroxymethyl)-1-methylpyrrolidin-3-yl)-4,6-dimethoxy-phenyl)-3-(pyridin-3-yl)prop-2-en-1-one,
(+/−)1-[2-Hydroxy-3-(2-hydroxymethyl-1-methyl-pyrrolidin-3-yl)-4,6-dimethoxy-phenyl]-3-(3-trifluoromethyl-phenyl)prop-2-en-1-one, and
   pharmaceutically acceptable salts or solvates thereof.

According to another aspect of the present invention there are provided processes for the preparation of the compounds of formula (I). Examples of processes for the preparation of the compounds of the present invention are described below and illustrated in schemes 1 to 5.

Compounds of formula (I) [denoted as (1d) and (1e) in Scheme 1], wherein R₃ is a group of formula (ii) as described herein above, may be prepared according to the process as illustrated in Scheme 1.

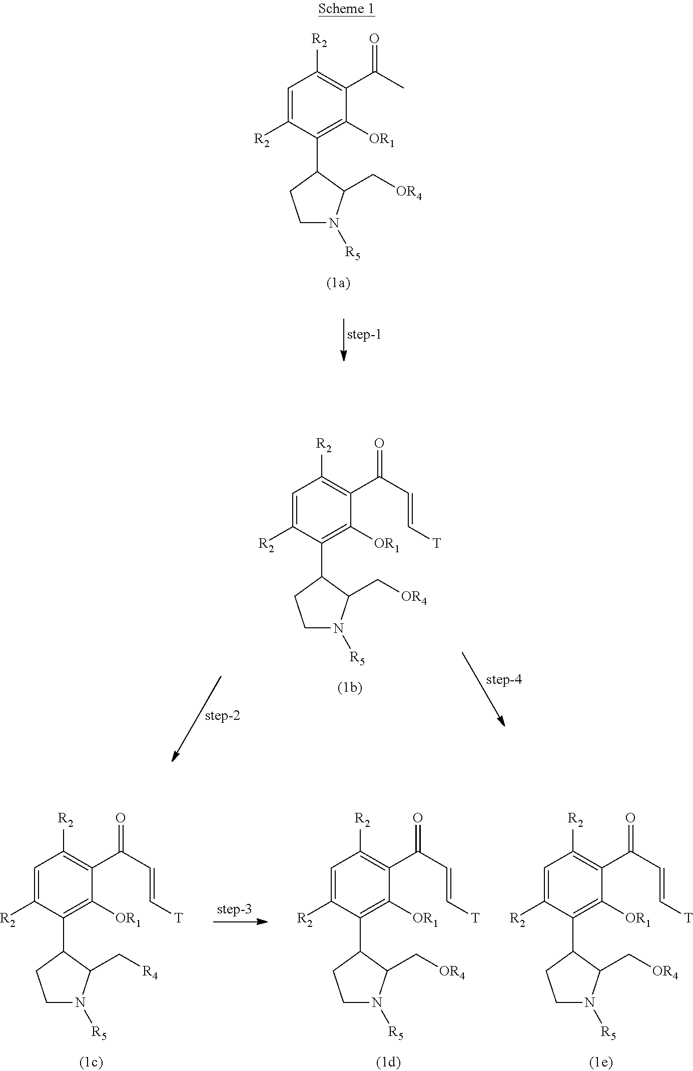

wherein,
R₁ is selected from hydrogen or alkyl;
R₂ at each occurrence is independently selected from hydrogen, halogen, hydroxy, alkyl or alkoxy;
R₄ is selected from hydrogen, halogen, alkyl or —C(O)-alkyl;
R₅ is selected from hydrogen or alkyl;
T is selected from phenyl or 5 or 6 membered heteroaryl;
wherein the phenyl and heteroaryl are unsubstituted or substituted by at least one group selected from halogen, hydroxy, alkyl, alkoxy, carboxy, amino, nitro or cyano.

### Step-1

A compound of formula (1a) (wherein R₁ is hydrogen) is obtained according to a method as described in PCT publication WO 2007148158. Compound of formula (1b) (wherein R₁ is hydrogen) is prepared by condensing compound of formula (1a) with a compound of a formula T-CHO (wherein T is selected from phenyl or 5 or 6 membered heteroaryl; wherein the phenyl and heteroaryl are unsubstituted or substituted by at least one group selected from halogen, hydroxy, alkyl, haloalkyl, alkoxy, carboxy, amino, nitro or cyano). The condensation may be carried out according to a method known to a person skilled in the art, such as the Claisen-Schmidt condensation (Synthesis, 1980, 8, 647-650; J. Med. Chem., 1995, 38, 5031) wherein compound of formula (1a) is condensed with a compound of formula T-CHO (wherein T is as defined above), in the presence of an aqueous alcoholic alkali wherein the alkali is selected from sodium hydroxide or potassium hydroxide to obtain the compound of formula (1b).

The condensation procedure as described herein above involves the use of a base and a solvent. The base is selected from an organic or inorganic base. The organic base is selected from triethylamine, pyridine, pyrrolidine, lutidine or a mixture thereof. The inorganic base is selected from sodium hydroxide, potassium hydroxide, sodium carbonate, sodium hydride, sodamide or n-butyllithium. The amount of base used may vary from 1 to 8 equivalents. The solvent is a protic or an aprotic solvent selected from diethyl ether, tetrahydrofuran, tetrahydropyran, dioxane, toluene, water, methanol, ethanol, dimethylformamide (DMF) or dimethyl sulfoxide (DMSO).

### Step-2

Compound of formula (1c) (wherein R₁ is hydrogen and R₄ is halogen) is prepared by reacting compound of formula (1b) (wherein R₁ is hydrogen and R₄ is hydrogen) with triphenyl phosphine in presence of carbon tetrachloride or carbon tetrabromide. Compound of formula (1c) (wherein R₁ is hydrogen and R₄ is halogen) can also be prepared by reacting compound of formula (1b) (wherein R₁ is hydrogen and R₄ is hydrogen) with a halogenating agent selected from thionyl chloride or thionyl bromide, in a solvent selected from tetrahydrofuran, dioxane or toluene or in the absence of a solvent; at a temperature in the range of 20° C. to reflux temperature.

### Step-3

Compound of formula (1d) (wherein R₁ is hydrogen and R₄ is alkyl) is prepared by substitution reaction of compound of formula (1c) (wherein R₁ is hydrogen and R₄ is halogen) with an alkoxide at a temperature in the range of 10° C. to reflux temperature; in presence of a solvent selected from ether, dioxane or toluene. The alkoxide is selected from sodium methoxide, potassium methoxide, sodium ethoxide, potassium ethoxide, sodium butoxide or potassium butoxide.

The hydroxy group can be converted into —O—C(O) alkyl by conventional methods.

### Step-4

Compound of formula (1e) (wherein R₁ is alkyl and R₄ is alkyl) is prepared by alkylating compound of formula (1b) (wherein R₁ is hydrogen and R₄ is hydrogen) with an alkylating agent in the presence of a base and a solvent at a temperature in the range of 10° C. to reflux temperature. The alkylating agent is selected from alkyl halide or dialkyl sulfide. The alkyl halide is selected from methyl iodide, or ethyl iodide. The dialkyl sulfide is dimethyl sulfide. The solvent is selected from acetone, ether, THF, dioxane, water or a mixture of water and an alcohol, selected from methanol, ethanol or propanol. The base is selected from organic and inorganic bases. The organic base is selected from triethylamine, or pyridine. The inorganic base is selected from sodium carbonate, potassium carbonate, or sodium hydride.

Compounds of formula (I) [denoted as (2m) and (2n) in Scheme 2], wherein R₃ is a group of formula (I) or (iii) as described herein above and R₄ is hydrogen, may be prepared according to the method illustrated in Scheme 2.

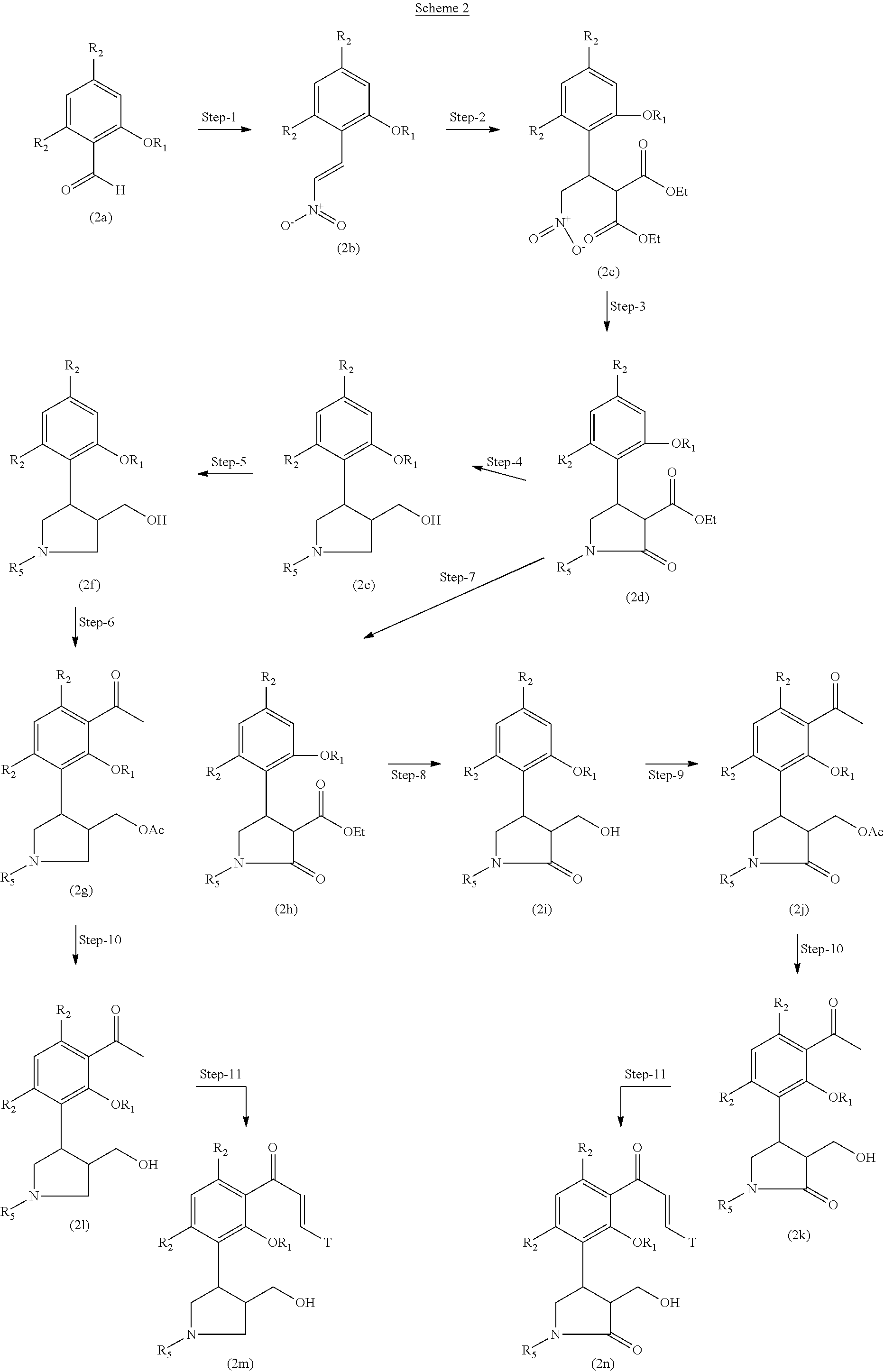

wherein:
R₁ is selected from hydrogen or alkyl;
R₂ at each occurrence is independently selected from hydrogen, halogen, hydroxy, alkyl or alkoxy;
R₅ is selected from hydrogen or alkyl;
T is selected from phenyl or 5 or 6 membered heteroaryl;
wherein the phenyl and heteroaryl are unsubstituted or substituted by at least one group selected from halogen, hydroxy, alkyl, alkoxy, carboxy, amino, nitro or cyano.

### Step-1

Compound of formula (2b) (wherein R₁ is alkyl) may be prepared by condensing a compound of formula (2a) (wherein R₁ is alkyl) with a nitroalkane. The condensation with nitroalkane is carried out in presence of acetic acid and sodium acetate or ammonium acetate; at a temperature in the range of 60° C. to reflux temperature.

### Step-2

Compound of formula (2c) (wherein R₁ is alkyl) is prepared by Michael addition of diethyl malonate to the compound of formula (2b) in presence of a base and a solvent at a temperature in the range of 0° C.-40° C.

The base is selected from sodium hydride, sodium alkoxide or potassium alkoxide. The solvent is selected from an ether or an alcohol, The ether may be selected from tetrahydrofuran or dioxane, and the alcohol from methanol or ethanol

### Step-3

Compound of formula (2d) (wherein R₁ is alkyl and R₅ is hydrogen) is prepared by reductive cyclization of the compound of formula (2c) using a reducing agent selected from Raney nickel and hydrogen at a pressure of 40 psi or iron/ammonium chloride (Fe/NH₄Cl) in methanol or iron/acetic acid (Fe/CH₃COOH) at a temperature in the range of 25° C. to reflux temperature.

### Step-4

Compound of formula (2e) (wherein R₁ is alkyl and R₅ is hydrogen) is obtained by reduction of compound of formula (2d) using a reagent selected from sodium cyanoborohydride, sodium triacetoxy borohydride, lithium aluminum hydride, borane in tetrahydrofuran or borane dimethyl sulfide in a solvent selected from diethyl ether, tetrahydrofuran or dioxane at reflux temperature.

### Step-5

Compound of formula (2f) (wherein R₁ is alkyl and R₅ is methyl) is obtained by N-alkylation of the compound of formula (2e), by hydrogenation using 10% palladium on charcoal in presence of formalin, in methanol at a temperature in the range of 20-55° C. and pressure in the range of 40-60 psi.

### Step-6

Compound of formula (2g) (wherein R₁ is hydrogen and R₅ is methyl) is obtained by reacting compound of formula (2f) with an acylating agent in the presence of a Lewis acid and a solvent at a temperature in the range of 0° C. to 40° C. The acylating agent is selected from acetic anhydride and acetyl chloride. The Lewis acid is selected from aluminium chloride (AlCl₃), zinc chloride (ZnCl₂), zinc bromide (ZnBr₂) or boron trifluoride etherate. The solvent is a chlorinated solvent selected from dichloromethane or chloroform.

### Step-7

Compound of formula (2h) (wherein R₁ is alkyl and R₅ are methyl) is obtained by reacting the compound of formula (2d) with an alkylating agent in presence of a base and a solvent at a temperature in the range of 0° C. to 40° C. The alkylating agent is selected from methyl iodide or dimethyl sulfate. The base is selected from sodium hydride or potassium tert-butoxide. The solvent is selected from diethyl ether, tetrahydrofuran, dioxane or aqueous alcohol. The alcohol is selected from methanol or ethanol.

### Step-8

Compound of formula (2i) (wherein R₁ is alkyl and R₅ are methyl) is obtained by reducing the compound of formula (2h) using sodium borohydride in refluxing alcohol selected from methanol, ethanol or butanol or mixtures thereof.

### Step-9

Compound of formula (2j) (wherein R₁ is hydrogen and R₅ is methyl) is obtained by reacting compound of formula (2i) with an acylating agent in the presence of a Lewis acid and a solvent at a temperature in the range of 0° C. to 40° C. The acylating agent is selected from acetic anhydride and acetyl chloride. The Lewis acid is selected from aluminium chloride (AlCl₃), zinc chloride (ZnCl₂), zinc bromide (ZnBr₂) or boron trifluoride etherate. The solvent is a chlorinated solvent selected from dichloromethane or chloroform.

### Step-10

Compounds of formula (2k) (wherein R₁ is hydrogen and R₅ is methyl) or (21) (wherein R₁ is hydrogen and R₅ is methyl) are obtained by deacylation of the compounds of formula (2j) or (2g) respectively either by acid or base hydrolysis; more preferably by base hydrolysis using alkali hydroxide in water or in alcohol selected from methanol and ethanol; at a temperature in the range of 10° C. to reflux temperature. The alkali hydroxide is selected from lithium hydroxide, sodium hydroxide, barium hydroxide or potassium hydroxide.

### Step-11

Compounds of formula (2m) (wherein R₁ is hydrogen and R₅ is methyl) or (2n) (wherein R₁ is hydrogen and R₅ is methyl) are obtained by condensing the compounds of formula (2k) or (21) respectively with a compound of formula T-CHO (wherein T is substituted or unsubstituted phenyl or substituted or unsubstituted 5 or 6 membered heteroaryl). The condensation may be carried out according to a method known to a person skilled in the art such as that described in step-1 of scheme-1 as given herein above.

Compounds of formula (I) [denoted as (3g) and (3h) in Scheme 3], wherein R₃ is a group of formula (Iv) as described herein above, may be prepared according to the method illustrated in Scheme 3.

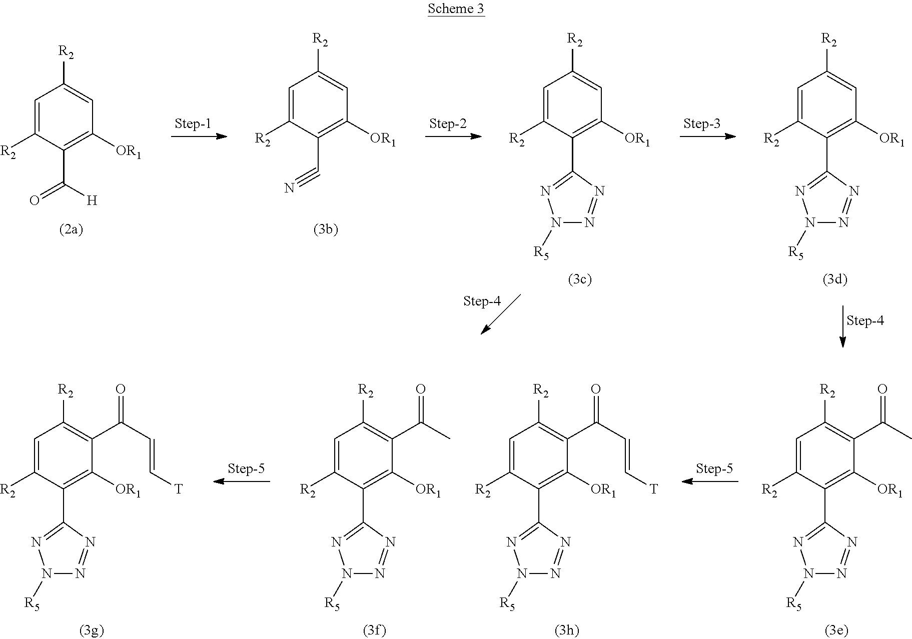

wherein:
R₁ is selected from hydrogen or alkyl;
R₂ at each occurrence is independently selected from hydrogen, halogen, hydroxy, alkyl or alkoxy;
R₅ is selected from hydrogen or alkyl;
T is selected from phenyl or 5 or 6 membered heteroaryl;
wherein the phenyl and heteroaryl are unsubstituted or substituted by at least one group selected from halogen, hydroxy, alkyl, alkoxy, carboxy, amino, nitro or cyano.

### Step-1

Compound of formula (3b) (wherein R₁ is alkyl) is obtained by converting the compound of formula (2a) into an aldoxime followed by dehydration using either an acid at a temperature in the range of 35° C. to reflux temperature or treating compound of formula (2a) with ammonia in the presence of iodine (J. Org. Chem., 2003, 68, 1158).

### Step-2

Compound of formula (3c) (wherein R₁ is alkyl and R₅ is hydrogen) is obtained by reacting compound of formula (3b) with an azide in the presence of a Lewis acid (J. Org. Chem., 2001, 66, 7945) in an aqueous medium under reflux temperature. The Lewis acid is selected from AlCl₃, ZnCl₂, ZnBr₂ or boron trifluoride etherate.

### Step-3

Compound of formula (3d) (wherein R₁ is alkyl and R₅ are methyl) is obtained by N-alkylation of compound of formula (3c) by hydrogenation using 10% palladium on charcoal in presence of formalin in methanol at a temperature in the range of 20° C. to 55° C. and a pressure of 40-60 psi.

### Step-4

Compounds of formula (3e) (wherein R₁ is hydrogen and R₅ is methyl) or (3f) (wherein R₁ and R₅ are hydrogen) are obtained by reacting compounds of formula (3d) or (3c) respectively with an acylating agent in the presence of a Lewis acid and a solvent at a temperature in the range of 0° C. to reflux condition.

The acylating agent is selected from acetic anhydride and acetyl chloride. The Lewis acid is selected from AlCl₃, ZnCl₂, ZnBr₂ or boron trifluoride etherate. The solvent is a chlorinated solvent selected from dichloromethane or chloroform.

### Step-5

Compounds of formula (3h) (wherein R₁ is hydrogen and R₅ is methyl) or (3g) (wherein R₁ and R₅ are hydrogen) are obtained by condensing compounds of formula (3e) or (3f) respectively with a compound of formula T-CHO (wherein T is substituted or unsubstituted phenyl or substituted or unsubstituted 5 or 6 membered heteroaryl). The condensation may be carried out according to a method known to a person skilled in the art such as that as described in step-1 of scheme 1 as given herein above.

Compounds of formula (I) [denoted as (4d) in Scheme 4], wherein R₃ is a group of formula (ii) as described herein above, may be prepared according to the process as illustrated in Scheme 4.

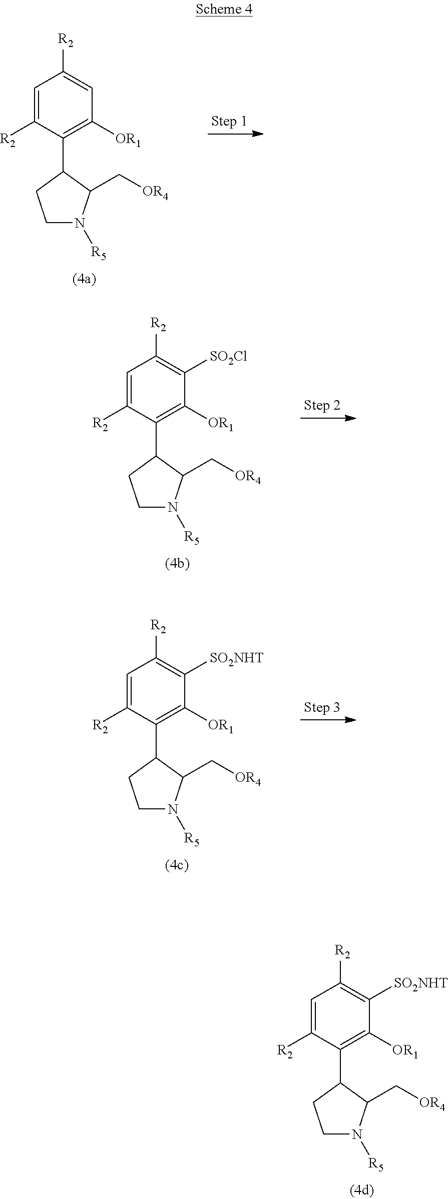

R₁ is selected from hydrogen or alkyl;
R₂ at each occurrence is independently selected from hydrogen, halogen, hydroxy, alkyl or alkoxy;
R₅ is selected from hydrogen or alkyl;
T is selected from phenyl or 5 or 6 membered heteroaryl;
wherein the phenyl and heteroaryl are unsubstituted or substituted by at least one group selected from halogen, hydroxy, alkyl, alkoxy, carboxy, amino, nitro or cyano.

### Step-1

Compound of formula (4a) (wherein R₁ is hydrogen or alkyl, R₂ is methoxy, R₄ is acetyl, and R₅ is hydrogen or alkyl) is synthesized as described in PCT publication WO2007148158. Compound of formula (4b) (wherein R₁ is hydrogen or alkyl, R₂ is methoxy, R₄ is acetyl, R₅ is hydrogen or alkyl) is obtained by reaction of the compound of formula (4a) with chlorosulfonic acid at a temperature in the range 0° C. to 100° C. Alternatively compound of formula (4b) is obtained by sulfonation of (4a) with sulfuric acid or oleum followed by reaction with excess of thionyl chloride at reflux condition (60° C. to 90° C.).

### Step-2

Compound of formula (4c) (wherein R₁ is hydrogen or alkyl, R₂ is methoxy, R₄ is acetyl, R₅ is hydrogen or alkyl, T is substituted phenyl or substituted heteroaryl) is obtained by reaction of (4b) (wherein R₁ is hydrogen or alkyl, R₂ is methoxy, R₄ is acetyl, R₅ is hydrogen or alkyl) with a primary amine (T-NH₂) (wherein T is substituted or unsubstituted phenyl or substituted or unsubstituted 5 or 6 membered heteroaryl) or in presence of an organic base selected from triethyl amine or N,N′-diisopropylethyl amine, in presence of a solvent selected from dichloromethane, or dichloroethane, at a temperature in the range 10° C. to 50° C.

### Step-3

Compound of formula (4d) (wherein R₁ is hydrogen or alkyl, R₂ is methoxy, R₄ is hydrogen, R₅ is hydrogen or alkyl, T is substituted or unsubstituted phenyl or substituted or unsubstituted 5 or 6 membered heteroaryl) is obtained by the hydrolysis of compound of formula (4c) (wherein R₁ is hydrogen or alkyl, R₂ is methoxy, R₄ is acetyl, R₅ is hydrogen or alkyl, T is substituted or unsubstituted phenyl or substituted or unsubstituted 5 or 6 membered heteroaryl) in presence of a base selected from lithium hydroxide (LiOH), sodium hydroxide (NaOH), and potassium hydroxide (KOH) in presence of mixture of solvent selected from methanol:water, THF:water, or ethanol:water, at a temperature in the range 20° C. to 60° C.

Compounds of formula (I) [denoted as (5d) in Scheme 5], wherein R₃ is a group of formula (ii) as described herein above, may be prepared according to the process as illustrated in Scheme 5.

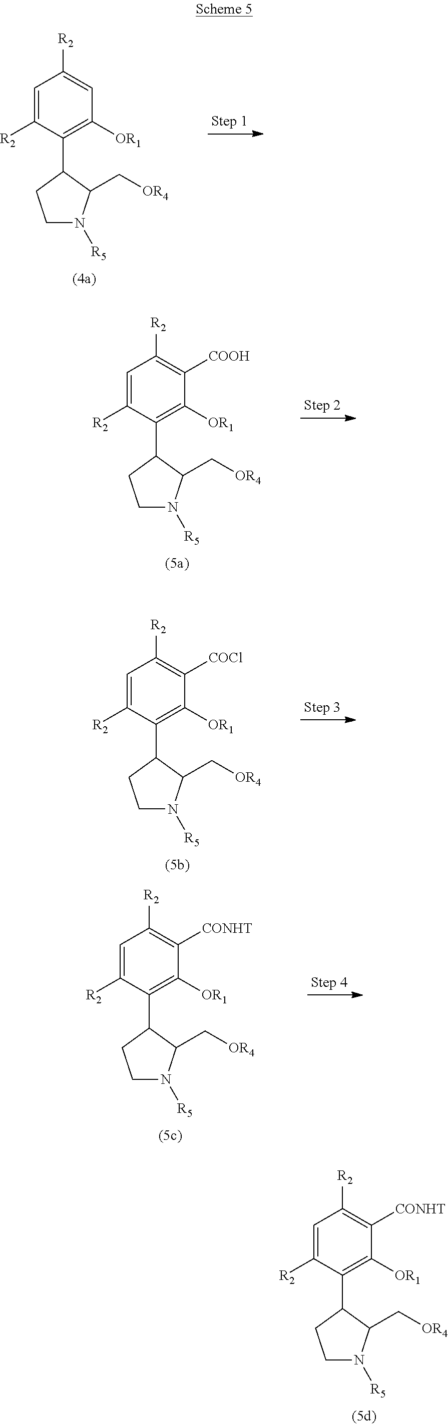

R₁ is selected from hydrogen or alkyl;
R₂ at each occurrence is independently selected from hydrogen, halogen, hydroxy, alkyl or alkoxy;
R₅ is selected from hydrogen or alkyl;
T is selected from phenyl or 5 or 6 membered heteroaryl;
wherein the phenyl and heteroaryl are unsubstituted or substituted by at least one group selected from halogen, hydroxy, alkyl, alkoxy, carboxy, amino, nitro or cyano.

### Step-1

Compound of formula (4a) (wherein R₁ is hydrogen or alkyl, R₂ is methoxy, R₄ is acetyl, R₅ is hydrogen or alkyl) synthesized as described in PCT publication WO2007148158. Compound of formula (5a) (wherein R₁ is hydrogen or alkyl, R₂ is methoxy, R₄ is acetyl, R₅ is hydrogen or alkyl) is obtained by lithiation of compound of formula (4a) by alkyl lithium such as 0.5 M to 2 M n-butyl lithium solution in a solvent selected from tetrahydrofuran, pentane, hexane, heptane, in an aprotic solvent selected from dry diethyl ether or dry tetrahydrofuran in an inert atmosphere (such as dry nitrogen or argon or helium) and at a temperature in the range −70° C. to +10° C. for 0.5 hour to 1 hour, followed by dry carbon dioxide (CO₂) gas circulation for a period of 2 hours to 3 hours.

Alternatively compound (5a) is also prepared by bromination of compound of formula (4a) either by bromine in acetic acid or bromine in chloroform or by N-bromosuccinimide to obtain bromo derivative of (4a) (wherein R₁ is hydrogen or alkyl, R₂ is methoxy, R₄ is acetyl, R₅ is hydrogen or alkyl) followed by lithium halogen exchange and dry carbon dioxide (CO₂) gas circulation for a period of 2 hours to 3 hours

### Step-2

Compound of formula (5b) (wherein R₁ is hydrogen or alkyl, R₂ is methoxy, R₄ is acetyl, R₅ is hydrogen or alkyl) is obtained by refluxing compound (5a) (wherein R₁ is hydrogen or alkyl, R₂ is methoxy, R₄ is acetyl, R₅ is hydrogen or alkyl) in a chlorinating agent such as thionyl chloride for 0.5 hour to 1 hour.

### Step-3

Compound of formula (5c) (wherein R₁ is hydrogen or alkyl, R₂ is methoxy, R₄ is acetyl, R₅ is hydrogen or alkyl, T is substituted or unsubstituted phenyl or substituted or unsubstituted 5 or 6 membered heteroaryl) can be obtained by treating compound of the formula (5b) (wherein R₁ is hydrogen or alkyl, R₂ is methoxy, R₄ is acetyl, R₅ is hydrogen or alkyl) with a primary amine (T-NH₂) (wherein T is substituted or unsubstituted phenyl or 5 or 6 substituted or unsubstituted membered heteroaryl) in presence of an organic base selected from triethyl amine, or N,N′-diisopropylethyl amine, in a solvent selected from dry dichloromethane, dichloroethane or dry tetrahydrofuran. Alternatively compound of the formula (5c) is obtained from compound of the formula (5a) by use of —COOH group activators or by the use of peptide coupling conditions as described in a reference Tetrahedron, 2004, 60, 11, 2447-67.

### Step-4

Compound of formula (5d) (wherein R₁ is hydrogen or alkyl, R₂ is methoxy, R₄ is hydrogen, R₅ is hydrogen or alkyl, wherein T is substituted or unsubstituted phenyl or 5 or 6 substituted or unsubstituted membered heteroaryl) is obtained by the hydrolysis of compound of formula (5c) (wherein R₁ is hydrogen or alkyl, R₂ is methoxy, R₄ is acetyl, R₅ is hydrogen or alkyl, T is phenyl or 5 or 6 membered heteroaryl) in presence of a base selected from lithium hydroxide (LiOH), sodium hydroxide (NaOH), or potassium hydroxide (KOH) in a mixture of solvents selected from methanol:water, tetrahydrofuran:water, or ethanol:water, at a temperature in the range 20° C. to 60° C.

The compounds of formula (I), as obtained in Schemes 1 to 5 may be optionally converted into their corresponding pharmaceutically acceptable salts.

It will be appreciated by those skilled in the art that the compounds of the present invention may also be utilized in the form of their pharmaceutically acceptable salts or solvates. Thus, when the compounds of the present invention represented by the general formula (I) contain one or more basic groups, i.e. groups which can be protonated, they can form an addition salt with an inorganic or organic acid. Examples of suitable inorganic acids include: boric acid, perchloric acid, hydrochloric acid, hydrobromic acid, hydrofluoric acid, sulfuric acid, sulfamic acid, phosphoric acid, nitric acid and other inorganic acids known to a person skilled in the art. Examples of suitable organic acids include: acetic acid, propionic acid, succinic acid, glycolic acid, stearic acid, lactic acid, malic acid, tartaric acid, citric acid, ascorbic acid, pamoic acid, maleic acid, hydroxymaleic acid, fumaric acid, phenylacetic acid, glutamic acid, benzoic acid, salicylic acid, sulfanilic acid, 2-acetoxybenzoic acid, toluenesulfonic acid, methanesulfonic acid, benzenesulfonic acid, ethane disulfonic acid, oxalic acid, isethionic acid, ketoglutaric acid, glycerophosphoric acid, aspartic acid, picric acid, lauric acid, palmitic acid, cholic acid, pantothenic acid, alginic acid, naphthoic acid, mandelic acid, tannic acid, camphoric acid and other organic acids known to a person skilled in the art.

Thus, when the compounds of the present invention represented by the general formula (I) contain an acidic group they can form an addition salt with a suitable base. For example, such salts of the compounds of the present invention may include their alkali metal salts such as Li, Na, and K salts, or alkaline earth metal salts such as Ca, Mg salts, or aluminium salts, or salts with ammonia or salts of organic bases such as lysine, arginine, guanidine, diethanolamine, choline, and tromethamine [tris(hydroxymethyl)aminomethane].

The pharmaceutically acceptable salts of the present invention can be synthesized from the subject compound, which contains a basic or an acidic moiety, by conventional chemical methods. Generally the salts are prepared by contacting the subject compound which may be a free base or acid with a desired salt-forming inorganic or organic acid or a base in a suitable solvent or dispersant or from another salt by cation or anion exchange. Suitable solvents are, for example, ethyl acetate, diethyl ether, methanol, ethanol, acetone, tetrahydrofuran, dioxane or mixtures of these solvents.

The present invention furthermore includes all solvates of the compounds of the formula (I), for example hydrates, and the solvates formed with other solvents of crystallization, methanol, ethanol, diethylether, ethyl acetate, dioxane, dimethylformamide (DMF), or acetone, or mixtures thereof.

The present invention also includes prodrugs thereof of compounds of formula (I) and their salts.

The compounds within the scope of the present invention find use in the treatment of a disease, condition or disorder mediated by one or more cytokines selected from TNF-α, IL-1, IL-6 or IL-8.

Conditions or disorders that may be treated by the compounds of formula (I) include, inflammatory bowel disease, inflammation, rheumatoid arthritis, juvenile rheumatoid arthritis, psoriatic arthritis, osteoarthritis, refractory rheumatoid arthritis, chronic non-rheumatoid arthritis, osteoporosis/bone resorption, Crohn's disease, septic shock, endotoxic shock, atherosclerosis, ischemia-reperfusion injury, coronary heart disease, vasculitis, amyloidosis, multiple sclerosis, sepsis, chronic recurrent uveitis, hepatitis C virus infection, malaria, ulcerative colitis, cachexia, psoriasis, plasmocytoma, endometriosis, Behcet's disease, Wegener's granulomatosis, AIDS, HIV infection, autoimmune disease, immune deficiency, common variable immunodeficiency (CVID), chronic graft-versus-host disease, trauma and transplant rejection, adult respiratory distress syndrome, pulmonary fibrosis, recurrent ovarian cancer, lymphoproliferative disease, refractory multiple myeloma, myeloproliferative disorder, diabetes, juvenile diabetes, meningitis, ankylosing spondylitis, skin delayed type hypersensitivity disorders, Alzheimer's disease, systemic lupus erythematosus and allergic asthma.

In one embodiment the conditions or disorders that may be treated by the compounds of formula (I) include, inflammatory bowel disease, inflammation, rheumatoid arthritis, psoriatic arthritis, osteoarthritis, ankylosing spondylitis, osteoporosis/bone resorption, Crohn's disease, atherosclerosis, ulcerative colitis, and psoriasis.

In another embodiment the condition or disorder that may be treated by the compounds of formula (I) is, rheumatoid arthritis.

According to another aspect of the present invention there is provided a method for the treatment of a condition or a disorder mediated by one or more cytokines selected from TNF-α, IL-1, IL-6 or IL-8, comprising administering to a mammal in need thereof a therapeutically effective amount of one or more compound of formula (I).

### Pharmaceutical Compositions and Methods

In respect of the pharmaceutical compositions and medicaments reference to compounds of formula (I) includes stereoisomers, tautomeric forms, pharmaceutically acceptable salts, solvates and prodrugs thereof.

According to another aspect of the invention, there are provided pharmaceutical compositions comprising one or more compounds of formula (I) as active ingredients useful in the treatment of a condition or disorder mediated by one or more cytokines selected from TNF-α, IL-1, IL-6 or IL-8.

The pharmaceutical compositions and medicaments according to the invention are prepared in a manner known per se and familiar to a person skilled in the art. Pharmaceutically acceptable inert inorganic and/or organic carriers and/or additives can be used in addition to the compounds of formula (I), and/or their pharmaceutically acceptable salts. For the production of pills, tablets, coated tablets and hard gelatin capsules it is possible to use, for example, lactose, corn starch or derivatives thereof, gum arabica, magnesia or glucose, etc. Carriers for soft gelatin capsules and suppositories are, for example, fats, waxes, natural or hardened oils, etc. Suitable carriers for the production of solutions, for example injection solutions, or of emulsions or syrups are, for example, water, physiological sodium chloride solution or alcohols, for example, ethanol, propanol or glycerol, sugar solutions, such as glucose solutions or mannitol solutions, or a mixture of the various solvents which have been mentioned.

According to another aspect of the present invention there are provided methods for the manufacture of medicaments comprising one or more compounds of formula (I), which are useful for the treatment of a condition or disorder mediated by one or more cytokines selected from TNF-α, IL-1, IL-6 or IL-8.

The pharmaceutical compositions normally contain about 1 to 99% of compound of formula (I), for example, about 5 to about 70%, or from about 10 to about 30% by weight of the compound of the formula (I) and/or its salt. The amount of the active ingredient of the formula (I) and/or its salt in the pharmaceutical preparations normally is from about 5 to 500 mg.

The dose of the compounds of this invention which is to be administered will depend upon a variety of factors including the route of administration, the time of administration, the rate of excretion of the particular compound being employed, the duration of the treatment, other drugs, compounds and/or materials used in combination with the particular compounds employed, the age, sex, weight, condition, general health and prior medical history of the patient being treated, and like factors well known in the medical arts.

The dose to be administered daily is to be selected to produce the desired effect. A suitable dosage is about 1 to 100 mg/kg/day of the compound of formula (I) and/or salt, for example, about 1 to 50 mg/kg/day of a compound of formula (I) or a pharmaceutically acceptable salt of the compound. If required, higher or lower daily doses can also be administered. Actual dosage levels of the active ingredients in the pharmaceutical compositions of this invention may be varied so as to obtain an amount of the active ingredient, which is effective to achieve the desired therapeutic response for a particular patient, composition, and mode of administration without without excessive toxicity, irritation, allergic response, or other problem or complication, commensurate with a reasonable benefit/risk ratio to the patient.

The pharmaceutical compositions can be administered orally, for example in the form of pills, tablets, coated tablets, capsules, granules or elixirs. Administration can also be carried out parenterally, for example intravenously, intramuscularly or subcutaneously, in the form of injectable sterile solutions or suspensions, or topically, for example in the form of gels, creams or ointments or transdermally in the form of patches, or rectally, for example in the form of suppositories, or in other ways, for example in the form of aerosols or nasal sprays.

In addition to the active ingredient of the general formula (I) and/or its salt and carrier substances, the pharmaceutical preparations can contain additives such as, for example, fillers, antioxidants, dispersants, emulsifiers, defoamers, flavors, preservatives, solubilizers or colorants. They can also contain two or more compounds of the general formula (I) and/or their salts. Furthermore, in addition to at least one compound of the general formula (I) and/or its salt, the pharmaceutical preparations can also contain one or more other therapeutically or prophylactically active ingredients.

It is understood that modifications that do not substantially affect the activity of the various embodiments of this invention are included within the invention disclosed herein. Accordingly, the following examples are intended to illustrate but not to limit the present invention.

### EXAMPLES

The invention is further understood by reference to the following examples, which are intended to be purely exemplary of the invention. The present invention is not limited in scope by the exemplified embodiments, which are intended as illustrations of single aspects of the invention only. Any methods that are functionally equivalent are within the scope of the invention. Various modifications of the invention in addition to those described herein will become apparent to those skilled in the art from the foregoing description. Such modifications fall within the scope of the appended claims.

Unless otherwise stated all temperatures are in degree Celsius. Also, in these examples and elsewhere, abbreviations have the following meanings:

L: litre
ml: millilitre
μl: microlitre
gm: gram
mg: milligram
μg: microgram
mmol: millimole
μM: micromolar
N₂: nitrogen
CO₂: carbon dioxide
conc.: concentrated
anhy.: anhydrous
aq.: aqueous
HCl: hydrochloric acid
NaHCO₃: sodium bicarbonate
NaOH: sodium hydroxide
NaBH₄: sodium borohydride
Na₂CO₃: sodium carbonate
Na₂SO₄: sodium sulphate
DBTA: dibenzoyl tartaric acid
EDTA: ethylenediaminetetraacetic acid
THF: tetrahydrofuran
EtOAc: ethyl acetate
CH₂Cl₂: dichloromethane
CHCl₃: chloroform
MeOH: methanol
DMF: dimethylformamide
DMSO: dimethyl sulphoxide
DMAP: 4-dimethylaminopyridine
RT: room temperature (25±5° C.)

### Example 1

### 1-Methyl-4-(2,4,6-trimethoxy-phenyl)-1,2,3,6-tetrahydropyridine

To a solution of 1,3,5-trimethoxy-benzene (950 gm, 5.6×10³ mmol) in glacial acetic acid (1000 ml) was slowly added 1-methyl-4-piperidone (460 gm, 4×10³ mmol). To the reaction mixture, conc. HCl (600 ml) was added over a period of 20 minutes, at about 40° C. The temperature was raised to 85-90° C. and the reaction mixture was stirred for 3.5 hours. The reaction mixture was cooled to 40° C., poured over crushed ice (4 kg) and stirred for 20 minutes. The unreacted 1,3,5-trimethoxy-benzene was filtered and the filtrate was cooled below 10° C. The pH of the filtrate was adjusted to 11-12 using 50% aq. NaOH solution, the resultant solid was filtered, washed with water and dried to obtain the title compound.

Yield: 682 gm (75%); ¹HNMR (CDCl₃): δ 6.1 (s, 2H), 5.6 (m, 1H), 3.9 (s, 6H), 3.76 (s, 3H), 3.1 (t, 2H), 2.7 (t, 2H), 2.4 (s, 3H), 2.3 (m, 2H); MS: m/e 264 (M+1).

### Example 2

### (+/−)-trans-1-Methyl-4-(2,4,6-trimethoxy-phenyl)piperidin-3-ol

To a solution of compound of example 1 (400 gm, 1.52×10³ mmol) and NaBH₄ (120 gm, 3.24×10³ mmol) in dry THF (2.5 L) was added boron trifluoride etherate (400 ml, 3.15×10³ mmol) slowly with stirring, under N₂ atmosphere, at a temperature of about 0° C. The temperature of the reaction mixture was raised to 55° C., and the mixture stirred for 1.5 hours. The reaction mixture was cooled to 30° C., ice cold water (100 ml) was slowly added followed by conc. HCl (450 ml). The reaction mixture was stirred for 1 hour at a temperature in the range of 50-55° C., cooled to 30° C. and pH was adjusted to 11-12 using 50% aq. NaOH solution. Hydrogen peroxide (30%, 250 ml) was added over a period of 0.5 hours to the reaction mixture, and the mixture was stirred at 55-60° C. for 1.5 hours. The mixture was cooled to 30° C., followed by addition of water to dissolve the precipitated salts. The organic layer was separated and the aqueous layer was extracted using EtOAc (1 L×2). The organic layers were combined and dried (anhy. Na₂SO₄) and concentrated to obtain crude viscous brown oil. The oil was treated with 4N HCl (1 L) and extracted using EtOAc (500 ml×2). The aqueous layer was cooled, followed by addition of 50% aq. NaOH solution and extracted using EtOAc (500 ml×2). The organic layer was dried (anhy. Na₂SO₄) and was concentrated to obtain the title compound.

Yield: 231 gm (54%); ¹HNMR (CDCl₃): δ 6.13 (s, 2H), 4.35 (m, 1H), 3.77-3.79 (2s, 9H), 3.18 (m, 1H), 3.08 (m, 1H), 2.87 (d, 1H), 2.40 (s, 3H), 2.03 (m, 2H), 1.85 (t, 1H), 1.54 (m, 2H); MS: m/e 282 (M+1).

### Example 3

### (+/−)-trans-Acetic acid-1-methyl-3-(2,4,6-trimethoxy-phenyl)-pyrrolidin-2-yl methyl ester

To a solution of compound of example 2 (188 gm, 0.66×10³ mmol) in dry CH₂Cl₂ (1000 ml) was added distilled triethylamine (186 ml, 1.33×10³ mmol) slowly, followed by addition of methanesulfonyl chloride (62.5 ml, 0.8×10³ mmol) under stirring, at 0° C., under N₂ atmosphere over a period of 20 minutes. The reaction mixture was further stirred for 1 hour at 0° C., and was poured in saturated aq. NaHCO₃ solution (1 L). The organic layer was separated, washed with brine, dried (anhy. Na₂SO₄) and was concentrated to obtain O-mesylated derivative. To a solution of the O-mesylated derivative in distilled isopropyl alcohol (800 ml), was added anhydrous sodium acetate (219 gm, 2.6×10³ mmol) and the reaction mixture was refluxed for 1 hour. The reaction mixture was cooled to room temperature, filtered and washed with EtOAc. The filtrate was concentrated, and the crude product obtained was purified by column chromatography (silica gel, 50% EtOAc in hexane) to obtain the title compound.

Yield: 90 gm (41.6%); ¹HNMR (CDCl₃): δ 6.11 (s, 2H), 4.0 (t, 2H), 3.76-3.79 (2s, 9H), 3.77 (m, 1H), 3.14 (m, 1H), 2.63 (m, 2H), 2.41 (s, 3H), 2.03 (m, 2H), 1.99 (s, 3H); MS: m/e 324 (M+1).

### Example 4

### (+/−)-trans-(1-Methyl-3-(2,4,6-trimethoxy-phenyl)pyrrolidin-2-yl)methanol

To a solution of compound of example 3 (90 gm, 0.27×10³ mmol) in methanol (223 ml) was added 10% aq. NaOH solution (223 ml). The reaction mixture was stirred at 50° C. for 45 minutes, concentrated and then poured into ice water (500 ml). The reaction mixture was extracted using EtOAc (500 ml×2), washed with brine and dried (anhy. Na₂SO₄). The solvent was evaporated to obtain the title compound.

Yield: 73 gm (93%); ¹HNMR (CDCl₃): δ 6.14 (s, 2H), 3.89 (m, 1H), 3.80 (2s, 9H), 3.56 (dd, 1H), 3.88 (m, 1H), 3.15 (m, 1H), 2.72 (bs, 1H), 2.69 (m, 1H), 2.5 (m, 1H), 2.35 (s, 3H), 1.92 (m, 2H); MS: m/e 282 (M+1).

### Example 5A

### (−)-trans-(1-Methyl-3-(2,4,6-trimethoxy-phenyl)pyrrolidin-2-yl)methanol

To a solution of compound of example 4 (70 gm, 0.24×10³ mmol) in methanol (100 ml) heated to 70° C., was added (+) DBTA (90 gm, 0.25×10³ mmol) and the heating was continued for 10 minutes. The reaction mixture was concentrated to obtain a solid (160 gm), which was crystallized using methanol (160 ml) and isopropanol (1600 ml), filtered and dried to obtain crystalline tartarate salt (75 gm). The salt was recrystallized using methanol (75 ml) and isopropyl alcohol (750 ml). To a suspension of the salt (10 gm) in EtOAc (100 ml) was added 5% aq. NaHCO₃ (100 ml) and the mixture was stirred for 30 minutes. The organic layer was separated and the aqueous layer was further extracted using EtOAc (50 ml×2). The organic layers were combined and concentrated to obtain the title compound.

Yield: 3.65 gm (21%); [α]_{D}²⁵=−17.25° (c=0.98, methanol); ¹HNMR (CDCl₃): δ 6.15 (s, 2H), 3.92 (m, 1H), 3.8 (2s, 9H), 3.6 (dd, 1H), 3.2 (m, 1H), 2.78 (m, 1H), 2.42 (s, 3H), 2.0 (m, 2H); MS: m/e 282 (M+1).

### Example 5B

### (+)-trans-[1-Methyl-3-(2,4,6-trimethoxy-phenyl)-pyrrolidin-2-yl]-methanol

Resolution of (+/−)-trans-[1 methyl-3-(2,4,6-trimethoxy-phenyl)-pyrrolidin-2-yl]-methanol (compound of example 4) was carried out by the procedure described in example 5A by using (−) DBTA.

Yield: 3.48 g (19%); [α]_{D}²⁵=+17.52° (c=0.94, methanol); ¹HNMR (CDCl₃): δ 6.14 (s, 2H), 3.91 (m, 1H), 3.79 (two singlets, 9H), 3.61 (dd, 1H), 3.19 (m, 1H), 2.77 (m, 1H), 2.41 (s, 3H), 1.99 (m, 2H); MS: m/e 282 (M+1).

### Example 6A

### (−)-trans-1-[2-Hydroxy-3-(2-hydroxymethyl-1-methyl-pyrrolidine-3-yl)-4,6-dimethoxy-phenyl]-ethanone

To a solution of compound of example 5A (15 gm, 0.05×10³ mmol) in acetic anhydride (27 ml, 0.26×10³ mmol) was added boron trifluoride etherate (33.5 ml, 0.26×10³ mmol) dropwise, with stirring at 0° C. under N₂ atmosphere. The reaction mixture was stirred at room temperature for 2 hours, poured over crushed ice (1 kg) and pH adjusted to alkaline by adding saturated aq. Na₂CO₃ solution. The reaction mixture was extracted using CHCl₃ (200 ml×3). The organic layer was washed using brine, dried (anhy. Na₂SO₄), concentrated and dissolved in MeOH (40 ml). To this was added 10% aq. NaOH (40 ml) and stirred at 50° C. for 1 hour. The reaction mixture was cooled to 10° C. followed by addition of 1N HCl, stirred for 5 minutes and made alkaline by adding saturated aq. Na₂CO₃ solution. The precipitate obtained was filtered, washed with water, and dried to obtain the title compound,

Yield: 9.7 gm (59%); [α]_{D}²⁵=−7.1° (c=0.68, methanol). ¹HNMR (CDCl₃): δ 5.94 (s, 1H), 3.9 (m, 1H), 3.88 (s, 3H), 3.87 (s, 3H), 3.55 (dd, 1H), 3.34 (m, 1H), 3.12 (m, 1H), 2.72 (m, 2H), 2.59 (s, 3H), 2.53 (m, 1H), 2.33 (s, 3H), 1.96 (m, 2H); MS: m/e 310 (M+1).

### Example 6B

### (+)-trans-1-[2-Hydroxy-3-(2-hydroxymethyl-1-methyl-pyrrolidine-3-yl)-4,6-dimethoxy-phenyl]-ethanone

To a solution of compound of example 5B (15 gm, 0.05×10³ mmol) in acetic anhydride (27 ml, 0.26×10³ mmol) was added boron trifluoride etherate (33.5 ml, 0.26×10³ mmol) drop wise, with stirring at 0° C. under N₂ atmosphere. The reaction mixture was stirred at room temperature for 2 hours, poured over crushed ice (1 kg) and pH adjusted to alkaline by adding saturated aq. Na₂CO₃ solution. The reaction mixture was extracted using CHCl₃ (200 ml×3). The organic layer was washed using brine, dried (anhy. Na₂SO₄), concentrated and dissolved in MeOH (40 ml). To this was added 10% aq. NaOH (40 ml) and stirred at 50° C. for 1 hour. The reaction mixture was cooled to 10° C. followed by addition of 1N HCl, stirred for 5 minutes and made alkaline by adding saturated aq. Na₂CO₃ solution. The precipitate obtained was filtered, washed with water, and dried to obtain the title compound.

Yield: 8.2 g (49.69%); [α]_{D}²⁵=+6.9° (c=0.70, methanol); ¹HNMR (CDCl₃): δ 5.93 (s, 1H) 3.88 (m, 1H) 3.87 (s, 3H) 3.85 (s, 3H) 3.54 (dd, 1H) 3.32 (m, 1H) 3.10 (m, 1H) 2.7 (m, 2H) 2.57 (s, 3H) 2.51 (m, 1H), 2.31 (s, 3H), 1.95 (m, 2H); MS: m/e 310 (M+1).

### Example 7

### (+/−)-trans-1-(2-Hydroxy-3-(2-(hydroxymethyl)-1-methylpyrrolidin-3-yl)-4,6-dimethoxy-phenyl)ethanone

To a solution of compound of example 4 (30 gm, 0.1×10³ mmol) in acetic anhydride (50 ml, 0.53×10³ mmol) was added boron trifluoride etherate (67 ml, 0.53×10³ mmol), with stirring at 0° C. under N₂ atmosphere. The reaction mixture was stirred at room temperature for 3 hours, poured over crushed ice (200 gm) and made alkaline by adding saturated aq. Na₂CO₃ solution. The reaction mixture was extracted using CHCl₃ (200 ml×3). The organic layer was washed using brine, dried (anhy. Na₂SO₄), concentrated and dissolved in MeOH (80 ml). To the reaction mixture was added 10% aq. NaOH (80 ml) and stirred at 50° C. for 1 hour. The reaction mixture was cooled to 10° C., followed by addition of 1N HCl, stirred for 5 minutes and made alkaline by adding saturated aq. Na₂CO₃ solution. The precipitate obtained was filtered, washed with water, and dried to obtain the title compound.

Yield: 10.8 gm (65%); ¹HNMR (CDCl₃): δ 5.94 (s, 1H), 3.98 (m, 1H), 3.91 (two singlet, 6H), 3.58 (dd, 1H), 3.37 (m, 1H), 3.14 (m, 1H), 2.79 (m, 2H), 2.6 (s, 3H), 2.58 (m, 1H), 2.38 (s, 3H), 2.01 (m, 2H); MS: m/e 310 (M+1).

### Example 8

### 2,4,6-Trimethoxy-benzaldehyde

2,4,6-Trimethoxy-benzene (40 gm, 0.22×10³ mmol) was added to dimethylformamide and stirred at a temperature in the range of −5 to 0° C. under N₂ atmosphere, followed by addition of phosphorus oxychloride (48 gm, 0.5×10³ mmol) drop wise over a period of 30-45 minutes. The reaction mixture was stirred for one hour at 0° C., poured over crushed ice followed by saturated sodium carbonate solution. Precipitate obtained was filtered and washed with water to obtain the title compound.

Yield: 46 gm (98%); ¹HNMR (CDCl₃): δ 10.35 (s, 1H), 6.67 (s, 2H), 3.88 (s, 6H), 3.87 (s, 3H); MS: m/e 197 (M+1).

### Example 9

### 1,3,5-Trimethoxy-2-(2-nitrovinyl)benzene

To a mixture of compound of example 8 (25 gm, 0.12×10³ mmol) and ammonium acetate (19.7 gm, 0.24×10³ mmol) in acetic acid stirred at room temperature was added nitromethane (11.8 ml, 0.21×10³ mmol), and the mixture was heated at 100° C. for 1.5 hours. The reaction mixture was cooled to room temperature and poured over crushed ice. The yellow precipitate obtained was filtered and washed with water to obtain the title compound.

Yield: 22 gm (72%); ¹HNMR (CDCl₃): δ 8.53 (d, 1H), 8.0 (d, 1H), 6.11 (s, 2H), 3.90 (s, 6H), 3.87 (s, 3H); MS: m/e 240 (M+1).

### Example 10

### 2-[2-Nitro-1-(2,4,6-trimethoxy-phenyl)-ethyl]-malonic acid diethyl ester

Dry ethanol (50 ml) was cooled to 10° C. and to it sodium metal pieces were added (0.28 gm, 6.20 mmol) under stirring, followed by addition of compound of example 9 (2 gm, 10.2 mmol) and diethylmalonate (1 ml, 12.5 mmol) at room temperature. The reaction mixture was stirred at room temperature for 1 hour, followed by addition of acetic acid and the stirring was continued further for 10 minutes. The solvent was evaporated, chloroform was added, washed with water, concentrated to obtain the crude product, which was purified by column chromatography (silica gel, 10% EtOAc in hexane) to obtain the title compound.

Yield: 2.5 gm (80%); ¹HNMR (CDCl₃): δ 6.06 (s, 2H), 4.88-4.81 (m, 3H), 4.25-4.17 (m, 3H), 3.89 (q, 2H), 3.80 (s, 6H), 3.77 (s, 3H), 1.28 (t, 3H), 0.98 (t, 3H); MS: m/e 400 (M+1).

### Example 11

### 2-Oxo-4-(2,4,6-trimethoxy-phenyl)pyrrolidine-3-carboxylic acid ethyl ester

To a solution of compound of example 10 (2 gm, 5 mmol) in methanol was added Raney nickel (0.23 gm) and the reaction mixture was hydrogenated at a pressure of 40 psi at room temperature for 2 hours. The reaction mixture was filtered through celite, filtrate was concentrated, and the solid obtained was crystallized using 20% EtOAc in hexane to obtain the title compound. Yield: 1.4 gm (98%). Alternatively, to a mixture of compound of example 10 (6 gm, 15 mmol) and iron powder (6 gm) charged with water (24 ml) was added acetic acid (24 ml, 0.4×10³ mmol) drop wise under stirring at 80° C. for 30 minutes. The reaction mixture was stirred further for 2 hours, cooled to room temperature, made alkaline by adding saturated aq. Na₂CO₃ solution and extracted using EtOAc (100 ml×3), concentrated to obtain the crude product, which was purified by column chromatography (silica gel, 10% EtOAc in hexane) to obtain the title compound.

Yield: 3.5 gm (73%); ¹HNMR (CDCl₃): δ 6.13 (s, 2H), 4.7 (q, 1H), 4.2 (m, 2H), 3.88 (m, 1H), 3.80 (s, 3H), 3.79 (s, 6H), 3.49 (q, 2H), 1.25 (t, 3H); MS: m/e 324 (M+1).

### Example 12

### (4-(2,4,6-Trimethoxy-phenyl)pyrrolidine-3-yl)methanol

To a solution of compound of example 11 (1 gm, 3 mmol) in dry THF (50 ml) under N₂ atmosphere, was added borane (2.4 gm, 30 mmol) in THF and the reaction mixture was refluxed for 14 hours. The reaction mixture was cooled to 0° C., water was added carefully, followed by addition of 1:1 aq. HCl. The reaction mixture was stirred at 50° C. for 30 minutes. The reaction mixture was cooled, followed by addition of saturated Na₂CO₃ solution and extracted using chloroform. Solvent was evaporated and the crude product obtained was purified by column chromatography (silica gel, 3-5% MeOH and 1% liquor ammonia in CHCl₃) to obtain the title compound.

Yield: 0.5 gm (51%); ¹HNMR (CDCl₃): δ 6.11 (s, 2H), 3.95 (m, 2H), 3.78-3.77 (two singlet, 9H), 3.56 (m, 2H), 3.3 (m, 1H), 3.2 (t, 1H), 3.18 (t, 1H), 2.8 (t, 1H), 2.5 (m, 1H); MS: m/e 268 (M+1).

### Example 13

### (1-Methyl-4-(2,4,6-trimethoxy-phenyl)pyrrolidin-3-yl)methanol

To a solution of compound of example 12 (5 gm 18.72 mmol) in methanol (50 ml) was added formalin (1.5 gm 56.17 mmol) and 10% Palladium on charcoal under N₂ atmosphere. The reaction mixture was hydrogenated at a pressure of 40 psi at room temperature for 3 hours, filtered through celite and solvent concentrated under reduced pressure. The crude product obtained was purified by column chromatography (silica gel, 5% MeOH and 1% liquor ammonia in CHCl₃) to obtain the title compound.

Yield: 5 gm (96%); ¹HNMR (CDCl₃): δ 6.13 (s, 2H), 3.95 (m, 2H), 3.81 (s, 6H), 3.79 (s, 3H), 3.78 (m, 1H), 3.61 (m, 2H), 3.23 (m, 3H), 2.69 (s, 3H); MS: m/e 282 (M+1).

### Example 14

### Acetic acid 4-(3-acetyl-2-hydroxy-4,6-dimethoxy-phenyl)-1-methyl-pyrrolidin-3-ylmethyl ester

To a solution of compound of example 13 (5 gm, 17.79 mmol) in acetic anhydride (8.8 ml, 88.96 mmol) was added boron trifluoride etherate (11.27 ml, 88.89 mmol) dropwise, with stirring at 0° C. under N₂ atmosphere. The reaction mixture was then stirred at room temperature for 2 hours, poured in crushed ice (0.2 kg) followed by addition of saturated aq. Na₂CO₃ solution. The reaction mixture was extracted using CHCl₃ (100 ml×3), and the organic layer was washed with brine, dried (anhy. Na₂SO₄) and concentrated to obtain the title compound, which was used directly for the preparation of compound of example 15. Yield: 3 gm (55%).

### Example 15

### 1-(2-Hydroxy-3-(4-(hydroxymethyl)-1-methylpyrrolidin-3-yl)-4,6-dimethoxy-phenyl)ethanone

To a solution of compound of example 14 (3 gm, 8.54 mmol) in methanol (6 ml) was added a 10% aqueous NaOH (6 ml, 17.94 mmol) solution with stirring at room temperature. The temperature of reaction mixture was raised to 50° C. for 45 minutes. The reaction mixture was then cooled to room temperature, followed by addition of concentrated HCl. The reaction mixture was concentrated to remove methanol, and made alkaline by adding saturated aq. Na₂CO₃ solution. The precipitate obtained was filtered, washed with water and dried to obtain the title compound.

Yield: 1.5 gm (56%); ¹HNMR (CDCl₃): δ 5.96 (s, 1H), 3.85-3.83 (s, 6H), 3.81 (m, 1H), 3.64 (m, 2H), 3.6 (m, 1H), 2.87 (m, 2H), 2.73 (m, 2H), 2.59 (s, 3H), 2.39 (s, 3H); MS: m/e 310 (M+1).

### Example 16

### 1-Methyl-2-oxo-4-(2,4,6-trimethoxy-phenyl)-pyrrolidine-3-carboxylic acid ethyl ester

To a cold (−10° C.) solution of compound of example 11 (1 gm, 3 mmol) in dry DMF (25 ml) under N₂ atmosphere was added sodium hydride (50%, 0.178 gm, 3.6 mmol) followed by addition of dimethyl sulphate (0.455 gm, 3.6 mmol). Temperature of the reaction mixture was allowed to rise to 25° C., and the mixture was stirred for another 1 hour. The reaction mixture was poured over crushed ice after adding 2-3 drops of methanol and extracted with EtOAc (100 ml×2). Solvent was evaporated and the crude product obtained was purified by column chromatography (silica gel 0.5-1% MeOH and 1% liquor ammonia in CHCl₃) to obtain the title compound.

Yield: 0.72 gm (72%); ¹HNMR (CDCl₃): δ 6.12 (5, 2H), 4.6 (q, 1H), 4.19 (m, 2H), 3.85 (m, 1H), 3.80 (s, 3H), 3.77 (s, 6H), 3.52 (t, 1H), 3.44 (t, 1H), 2.90 (s, 3H), 1.27 (t, 3H); MS m/e 338 (M+1).

### Example 17

### 3-(Hydroxymethyl)-1-methyl-4-(2,4,6-trimethoxy-phenyl)pyrrolidin-2-one

To a solution of compound of example 16 (2 gm, 6 mmol) in ethanol (80 ml) was added NaBH₄ (0.22 gm, 6 mmol) and refluxed for 8 hours. The reaction mixture was cooled to 25° C. and was purified by column chromatography (silica gel, 0.5-1% MeOH and 1% liquor ammonia in CHCl₃) to obtain the title compound.

Yield: 1.2 gm (70%); ¹HNMR (CDCl₃): δ 6.13 (s, 2H), 3.89 (s, 3H), 3.86 (s, 6H), 3.8 (m, 1H), 3.70 (m, 2H), 3.32 (m, 2H), 3.1 (m, 1H), 2.82 (s, 3H); MS: m/e 296 (M+1).

### Example 18

### Acetic acid (4-(3-acetyl-2-hydroxy-4,6-dimethoxy-phenyl)-1-methyl-2-oxo-pyrrolidin-3-ylmethyl ester

To a cold (0° C.) solution of compound of example 17 (0.6 gm, 2.2 mmol) in acetic anhydride (1.12 ml, 11 mmol) was added boron trifluoride etherate (1.4 ml, 11 mmol) drop wise. The reaction mixture was stirred at 25° C. for 1.5 hours, poured over crushed ice (50 gm), extracted using CHCl₃ (100 ml×2). Solvent was evaporated and the crude product obtained was purified by column chromatography (silica gel, 0.5-1% MeOH and 1% liquor ammonia in CHCl₃) to obtain the title compound.

Yield: 0.5 gm (71%); ¹HNMR (CDCl3): δ 14.2 (s, 1H), 5.96 (s, 1H), 4.25 (m, 2H), 4.03 (q, 1H), 3.9 (s, 3H), 3.88 (s, 3H), 3.65 (t, 1H), 3.37 (t, 1H), 3.27 (m, 1H), 2.91 (s, 3H), 2.61 (s, 3H), 1.98 (s, 3H); MS: m/e 324 (M⁺−42).

### Example 19

### 4-(3-Acetyl-2-hydroxy-4,6-dimethoxy-phenyl)-3-(hydroxymethyl)-1-methyl pyrrolidin-2-one

To a solution of compound of example 18 (1.5 gm, mmol) in methanol (2 ml), was added aq. NaOH (0.19 gm in 2 ml water), and the reaction mixture was stirred at 50° C. for 1.5 hours. The reaction mixture was then cooled to 10° C., 2% HCl was added, and the mixture was filtered and washed with water to obtain the title compound.

Yield: 0.99 gm (76%); ¹HNMR (CDCl₃): δ 14.0 (s, 1H), 5.97 (s, 1H), 3.91 (s, 3H), 3.88 (s, 3H), 3.86 (m, 1H), 3.72 (m, 2H), 3.35 (m, 2H), 3.15 (m, 1H), 2.89 (s, 3H), 2.62 (s, 3H); MS m/e 324 (M+1).

### Example 20

### (+/−)3-(2-Chlorophenyl)-1-(2-hydroxy-3-(2-(hydroxymethyl)-1-methylpyrrolidin-3-yl)-4,6-dimethoxy-phenyl)prop-2-en-1-one hydrochloride

### Step-1

### (+/−)3-(2-Chlorophenyl)-1-(2-hydroxy-3-(2-(hydroxymethyl)-1-methylpyrrolidin-3-yl)-4,6-dimethoxy-phenyl)prop-2-en-1-one

To a solution of compound of example 7 (1 gm, 3.2 mmol) in ethanol (10 ml) was added 20% aq. NaOH (10 ml) and stirred for 10 minutes. To this reaction mixture under N₂ atmosphere, 2-chlorobenzaldehyde (1.36 gm, 9.7 mmol) was added and stirred at 25° C. for 8 hours. The reaction mixture was then poured over crushed ice (25 gm), acidified with 10% aq. HCl followed by basification with saturated Na₂CO₃ solution. The reaction mixture was extracted with chloroform, and the organic layer was washed with water, dried (anhy. Na₂SO₄), and concentrated. The crude product obtained was purified by column chromatography (silica gel, mixture of 0.5-1% MeOH and 1% liquor ammonia in CHCl₃) to obtain the title compound.

Yield: 0.68 gm (49.27%); ¹HNMR (CDCl₃): δ 8.14 (d, 1H), 8.83 (d, 1H), 7.67 (m, 1H), 7.4 (m, 1H), 7.39 (m, 2H), 5.99 (s, 1H), 3.99 (m, 1H), 3.93-3.89 (s, 6H), 3.6 (dd, 1H), 3.5 (dd, 1H), 3.2 (m, 1H), 2.9 (q, 1H), 2.6 (m, 1H), 2.5 (s, 3H), 2.16 (m, 2H); MS: m/e 432 (M+1).

### Step-2

### Preparation of Hydrochloride Salt

To a cold solution of example 20 (0.1 gm, 0.23 mmol) in dry methanol was added 10% HCl in diethyl ether. It was stirred for 5 min. and evaporated under reduced pressure to obtain the title compound as pale yellow solid.

Yield: 0.1 gm (99%); ¹HNMR (CD₃OD): δ 8.15 (d, 1H), 7.79 (d, 1H), 7.8 (m, 1H), 7.42 (m, 1H), 7.39 (m, 2H), 6.25 (s, 1H), 4.2 (m, 1H), 4.02-3.99 (s, 6H), 3.8 (dd, 1H), 3.65 (dd, 1H), 3.6 (m, 1H), 3.58 (q, 1H), 3.45 (m, 1H), 3.01 (s, 3H), 2.42 (m, 1H), 2.2 (m, 1H); MS: m/e 432 (M+1).

### Example 20A

### (−)3-(2-Chlorophenyl)-1-(2-hydroxy-3-(2-(hydroxymethyl)-1-methylpyrrolidin-3-yl)-4,6-dimethoxy-phenyl)prop-2-en-1-one

The title compound was obtained by reaction of compound of example 6A (1 gm, 3.2 mmol) and 2-chlorobenzaldehyde (1.1 ml, 9.7 mmol) according to the procedure described in example 20, step 1.

Yield: 0.7 gm (50%); [α]_{D}²⁵=−21.28° (C=0.148 g/100 ml, methanol). ¹HNMR (CDCl₃): δ 8.12 (d, 1H), 8.87 (d, 1H), 7.65 (m, 1H), 7.39 (m, 1H), 7.36 (m, 2H), 6.01 (s, 1H), 4.1 (m, 1H), 3.93 (s, 3H), 3.89 (s, 3H), 3.58 (dd, 1H), 3.52 (dd, 1H), 3.19 (m, 1H), 2.87 (q, 1H), 2.61 (m, 1H), 2.4 (s, 3H), 2.02 (m, 2H); MS: m/e 432 (M+1).

### Example 20B

### (+)3-(2-Chlorophenyl)-1-(2-hydroxy-3-(2-(hydroxymethyl)-1-methylpyrrolidin-3-yl)-4,6-dimethoxy-phenyl)prop-2-en-1-one

The title compound was obtained by reaction of compound of example 6B (1 gm, 3.2 mmol) and 2-chlorobenzaldehyde (1.1 ml, 9.7 mmol) according to the procedure described in example 20, step 1.

Yield: 0.69 gm (48.1%); [α]_{D}²⁵=+24.65° (C=0.146 g/100 ml, methanol); ¹HNMR (CDCl₃): δ 8.13 (d, 1H), 8.25 (d, 1H), 7.61 (m, 1H), 7.4 (m, 1H), 7.38 (m, 2H), 5.98 (s, 1H), 3.98 (m, 1H), 3.93 (s, 3H), 3.88 (s, 3H), 3.55 (dd, 1H), 3.51 (dd, 1H), 3.2 (m, 1H), 2.81 (q, 1H), 2.58 (m, 1H), 2.39 (s, 3H), 2.01 (m, 2H); MS: m/e 432 (M+1).

### Example 21

### (+/−)3-(3-Bromophenyl)-1-(2-hydroxy-3-(2-(hydroxymethyl)-1-methylpyrrolidin-3-yl)-4,6-dimethoxy-phenyl)prop-2-en-1-one

The title compound was obtained by reaction of compound of example 7 (1 gm, 3.2 mmol) and 3-bromobenzaldehyde (1.13 ml, 9.7 mmol) according to the procedure described in example 20, step 1.

Yield: 0.89 gm (58%); ¹HNMR (CDCl₃): δ 7.83 (d, 1H), 7.78 (s, 1H), 7.72 (d, 1H), 7.5 (d, 2H), 7.27 (m, 1H), 6.0 (s, 1H), 3.99 (m, 1H), 3.96 (s, 3H), 3.91 (s, 3H), 3.59 (dd, 1H), 3.40 (m, 1H), 3.16 (m, 1H), 2.76 (m, 1H), 2.59 (m, 1H), 2.36 (s, 3H), 2.04 (m, 2H); MS: m/e 478 (M+1).

### Example 22

### (+/−)3-(2,4-Dimethoxy-phenyl)-1-(2-hydroxy-3-(2-(hydroxymethyl)-1-methyl pyrrolidin-3-yl)-4,6-dimethoxy-phenyl)prop-2-en-1-one

The title compound was obtained by reaction of compound of example 7 (1 gm, 3.2 mmol) and 2,4dimethoxybenzaldehyde (1.6 gm, 9.7 mmol) according to the procedure described in example 20, step 1.

Yield: 0.79 gm (53.5%); ¹HNMR (CDCl₃): δ 14.0 (bs, 1H), 8.11 (d, 1H), 7.88 (d, 1H), 7.55 (d, 1H), 6.46 (d, 1H), 6.0 (s, 1H), 5.96 (s, 1H), 3.97 (m, 1H), 3.96 (s, 3H), 3.91 (s, 3H), 3.90 (s, 3H), 3.87 (s, 3H), 3.6 (dd, 1H), 3.41 (m, 1H), 3.2 (m, 1H), 2.8 (m, 1H), 2.62 (m, 1H), 2.40 (s, 3H), 2.03 (m, 2H); MS: m/e 458 (M+1).

### Example 23

### (+/−)1-(2-Hydroxy-3-(2-(hydroxymethyl)-1-methylpyrrolidin-3-yl)-4,6-dimethoxy-phenyl)-3-m-tolylprop-2-en-1-one

The title compound was obtained by reaction of compound of example 7 (1 gm, 3.2 mmol) and m-tolualdehyde (1.15 ml, 9.7 mmol) according to the procedure described in example 20, step 1.

Yield: 0.64 gm (58%); ¹HNMR (CDCl₃): δ 14.0 (bs, 1H), 7.86 (d, 1H), 7.76 (d, 1H), 7.43 (d, 1H), 7.39 (s, 1H), 7.29 (t, 1H), 7.21 (d, 1H), 6.01 (s, 1H), 3.94 (s, 3H), 3.90 (s, 3H), 3.6 (dd, 1H), 3.41 (m, 1H), 3.16 (m, 1H), 2.79 (m, 1H), 2.60 (m, 1H), 2.39 (s, 3H), 2.36 (s, 3H), 2.02 (m, 2H); MS: m/e 412 (M+1).

### Example 24

### (+/−)1-(2-Hydroxy-3-(2-(hydroxymethyl)-1-methylpyrrolidin-3-yl)-4,6-dimethoxy-phenyl)-3-o-tolylprop-2-en-1-one

The title compound was obtained by reaction of compound of example 7 (1 gm, 3.2 mmol) and o-tolualdehyde (1.13 ml, 9.7 mmol) according to the procedure described in example 20, step 1.

Yield: 0.66 gm (50%); ¹HNMR (CDCl₃): δ 14.44 (bs, 1H), 8.09 (d, 1H), 7.79 (d, 1H), 7.26 (m, 4H), 6.01 (s, 1H), 3.97 (s, 3H), 3.45 (m, 1H), 3.91 (s, 3H), 3.66 (dd, 1H), 3.49 (m, 1H), 3.3 (m, 1H), 2.92 (m, 1H), 2.55 (m, 1H), 2.49 (s, 3H), 2.44 (s, 3H), 2.1 (m, 2H); MS: m/e 412 (M+1).

### Example 25

### (+/−)1-(2-Hydroxy-3-(2-(hydroxymethyl)-1-methylpyrrolidin-3-yl)-4,6-dimethoxy-phenyl)-3-(3-nitrophenyl)prop-2-en-1-one

The title compound was obtained by reaction of compound of example 7 (1 gm, 3.2 mmol) and 3-nitrobenzaldehyde (1.46 gm, 9.7 mmol) according to the procedure described in example 20, step 1.

Yield: 0.8 gm (56.3%); ¹HNMR (CDCl₃): δ 14.25 (bs, 1H), 8.46 (s, 1H), 8.25 (d, 1H), 7.96 (d, 1H), 7.90 (d, 1H), 7.76 (d, 1H), 7.59 (t, 1H), 6.02 (s, 1H), 3.99 (s, 3H), 3.94 (m, 1H), 3.92 (s, 3H), 3.65 (dd, 1H), 3.41 (m, 1H), 3.19 (m, 1H), 2.8 (m, 1H), 2.6 (m, 1H), 2.37 (s, 3H), 2.05 (m, 2H); MS: m/e 443 (M+1).

### Example 26

### (+/−)1-(2-Hydroxy-3-(2-(hydroxymethyl)-1-methylpyrrolidin-3-yl)-4,6-dimethoxy-phenyl)-3-(4-nitrophenyl)prop-2-en-1-one

The title compound was obtained by reaction of compound of example 7 (1 gm, 3.2 mmol) and 4-nitrobenzaldehyde (1.46 gm, 9.7 mmol) according to the procedure described in example 20, step 1.

Yield: 0.78 gm (55.2%); ¹HNMR (CDCl₃): δ 8.56 (s, 1H), 8.23 (d, 2H), 7.97 (d, 2H), 6.72 (s, 1H), 6.17 (s, 1H), 4.03 (m, 1H), 3.97 (s, 3H), 3.82 (s, 3H), 3.78 (dd, 1H), 3.64 (m, 1H), 3.30 (m, 1H), 3.21 (m, 1H), 3.08 (m, 1H), 2.77 (s, 3H), 2.3 (m, 2H); MS: m/e 443 (M+1).

### Example 27

### (+/−)3-(2-Bromophenyl)-1-(2-hydroxy-3-(2-(hydroxymethyl)-1-methylpyrrolidin-3-yl)-4,6-dimethoxy-phenyl)prop-2-en-1-one

The title compound was obtained by reaction of compound of example 7 (1 gm, 3.2 mmol) and 2-bromobenzaldehyde (1.12 ml, 9.7 mmol) according to the procedure described in example 20, step 1.

Yield: 0.81 gm (53%); ¹HNMR (CDCl₃): δ 8.1 (d, 1H), 7.79 (d, 1H), 7.68 (d, 1H), 7.64 (d, 1H), 7.36 (t, 1H), 7.24 (t, 1H), 6.0 (s, 1H), 3.96 (m, 1H), 3.94 (s, 3H), 3.91 (s, 3H), 3.63 (dd, 1H), 3.41 (m, 1H), 3.18 (m, 1H), 2.81 (m, 1H), 2.62 (m, 1H), 2.37 (s, 3H), 2.02 (m, 2H); MS: m/e 476, 478 (M+1).

### Example 28

### (+/−)3-(4-Chlorophenyl)-1-(2-hydroxy-3-(2-(hydroxymethyl)-1-methylpyrrolidin-3-yl)-4,6-dimethoxy-phenyl)prop-2-en-1-one

The title compound was obtained by the reaction of compound of example 7 (1 gm, 3.2 mmol) and 4-chlorobenzaldehyde (1.36 gm, 9.7 mmol) according to the procedure described in example 20, step 1.

Yield: 0.76 gm (55%); ¹HNMR (CDCl₃): δ 14.72 (bs, 1H), 7.85 (d, 1H), 7.75 (d, 1H), 7.54 (d, 2H), 7.39 (d, 2H), 6.02 (s, 1H), 4.12 (m, 1H), 3.96 (s, 3H), 3.92 (s, 3H), 3.8 (m, 1H), 3.7 (m, 2H), 3.3 (m, 1H), 3.01 (m, 3H), 2.62 (m, 1H), 2.2 (m, 2H); MS: m/e 432 (M+1).

### Example 29

### (+/−)3-(4-Fluorophenyl)-1-(2-hydroxy-3-(2-(hydroxymethyl)-1-methylpyrrolidin-3-yl)-4,6-dimethoxy-phenyl)prop-2-en-1-one

The title compound was obtained by reaction of compound of example 7 (1 gm, 3.2 mmol) and 4-fluoro benzaldehyde (1 ml, 9.7 mmol) according to the procedure described in example 20, step 1.

Yield: 0.69 gm (52%); ¹HNMR (CDCl₃): δ 7.76 (d, 1H), 7.74 (d, 1H), 7.59 (m, 2H), 7.12 (m, 2H), 6.01 (s, 1H), 3.96 (s, 3H), 3.95 (m, 1H), 3.91 (s, 3H), 3.62 (dd, 1H), 3.4 (m, 1H), 3.18 (m, 2H), 2.8 (m, 1H), 2.52 (m, 1H), 2.37 (s, 3H), 2.01 (m, 2H); MS: m/e 416 (M+1).

### Example 30

### (+/−)1-(2-Hydroxy-3-(2-(hydroxymethyl)-1-methylpyrrolidin-3-yl)-4,6-dimethoxy phenyl)-3-(4-methoxyphenyl)prop-2-en-1-one

The title compound was obtained by reaction of compound of example 7 (1 gm, 3.2 mmol) and p-anisaldehyde (1.2 ml, 9.7 mmol) according to the procedure described in example 20, step 1.

Yield: 0.65 gm (48%); ¹HNMR (CDCl₃): δ 7.76 (d, 1H), 7.72 (d, 1H), 7.57 (d, 2H), 6.94 (d, 2H), 6.01 (s, 1H), 3.95 (s, 3H), 3.93 (m, 1H), 3.90 (s, 3H), 3.89 (s, 3H), 3.62 (dd, 1H), 3.41 (m, 1H), 3.16 (m, 1H), 2.8 (m, 1H), 2.61 (m, 1H), 2.37 (s, 3H), 2.05 (m, 2H); MS: m/e 428 (M+1).

### Example 31

### (+/−)1-(2-Hydroxy-3-(2-(hydroxymethyl)-1-methylpyrrolidin-3-yl)-4,6-dimethoxy-phenyl)-3-p-tolylprop-2-en-1-one

The title compound was obtained by reaction of compound of example 7 (1 gm, 3.2 mmol) and p-tolualdehyde (1.14 ml, 9.7 mmol) according to the procedure described in example 20, step 1.

Yield: 0.74 gm (56%); ¹HNMR (CDCl₃): δ 7.85 (d, 1H), 7.78 (d, 1H), 7.51 (d, 2H), 7.22 (d, 2H), 6.01 (s, 1H), 3.95 (s, 3H), 3.93 (m, 1H), 3.90 (s, 3H), 3.62 (dd, 1H), 3.41 (m, 1H), 3.15 (m, 1H), 2.8 (m, 1H), 2.61 (m, 1H), 2.59 (s, 3H), 2.37 (s, 3H), 2.04 (m, 2H); MS: m/e 412 (M+1).

### Example 32

### (+/−)1-(2-Hydroxy-3-(2-(hydroxymethyl)-1-methylpyrrolidin-3-yl)-4,6-dimethoxy-phenyl)-3-phenylprop-2-en-1-one

The title compound was obtained by reaction of compound of example 7 (1 gm, 3.2 mmol) and benzaldehyde (0.99 ml, 9.7 mmol) according to the procedure described in example 20, step 1.

Yield: 0.6 gm (47%); ¹HNMR (CDCl₃): δ 7.88 (d, 1H), 7.79 (d, 1H), 7.62 (dd, 2H), 7.42 (m, 3H), 6.015 (s, 1H), 3.96 (s, 3H), 3.93 (m, 1H), 3.91 (s, 3H), 3.63 (dd, 1H), 3.41 (m, 1H), 3.17 (m, 1H), 2.8 (m, 1H), 2.6 (m, 1H), 2.37 (s, 3H), 2.02 (m, 2H); MS: m/e 398 (M+1).

### Example 33

### (+/−)3-(3-Aminophenyl)-1-(2-hydroxy-3-(2-(hydroxymethyl)-1-methylpyrrolidin-3-yl)-4,6-dimethoxy-phenyl)prop-2-en-1-one

To a mixture of compound of example 25 (0.1 gm, 0.2 mmol) and iron powder (0.1 gm) in methanol (10 ml) was added ammonium chloride (0.06 gm, 1.13 mmol) and stirred at reflux temperature for 8 hours. The mixture was then cooled to room temperature and filtered, and the crude product obtained was purified by column chromatography (silica gel, mixture of 0.5-1% MeOH and 1% liquor ammonia in CHCl₃) to obtain the title compound.

Yield: 0.053 gm (57%); ¹HNMR (CDCl₃): δ 7.72 (d, 1H), 7.70 (d, 1H), 7.5 (d, 2H), 6.9 (d, 2H), 6.01 (s, 1H), 3.93 (s, 3H), 3.92 (m, 1H), 3.90 (s, 3H), 3.6 (dd, 1H), 3.4 (m, 1H), 3.15 (m, 1H), 2.79 (m, 1H), 2.6 (m, 1H), 2.36 (s, 3H), 2.02 (m, 2H); MS: m/e 413 (M+1).

### Example 34

### (+/−)Acetic acid-(3-(3-(3-(2-chlorophenyl)acryloyl)-2-hydroxy-4,6-dimethoxy-phenyl)-1-methylpyrrolidin-2-ylmethyl ester

To a solution of compound of example 20, step 1 (0.1 gm, 0.23 mmol) and DMAP (5 mg) in dry CH₂Cl₂ (25 ml) was added acetic anhydride (0.28 ml, 0.27 mmol) dropwise and stirred at 25° C. for 0.5 hour. The reaction mixture was poured over crushed ice, made alkaline by adding saturated aq. Na₂CO₃ solution and extracted with EtOAc (3×200 ml). Solvent was evaporated and the crude product obtained was purified by column chromatography (silica gel, mixture of 0.5% MeOH and 1% liquor ammonia in CHCl₃) to obtain the title compound.

Yield: 0.09 gm (90%); ¹HNMR (CDCl₃): δ 8.15 (d, 1H), 7.85 (d, 1H), 7.71 (m, 1H), 7.42 (m, 1H), 7.3 (m, 2H), 5.98 (s, 1H), 3.94 (s, 3H), 3.90 (s, 3H), 3.68 (m, 1H), 3.47 (m, 1H), 3.28 (m, 1H), 2.97 (s, 3H), 2.93 (m, 1H), 2.61 (m, 1H), 2.18 (m, 2H), 2.02 (s, 3H); MS: m/e 474 (M+1).

### Example 35

### (+/−)3-(3-Fluorophenyl)-1-(2-hydroxy-3-(2-(hydroxymethyl)-1-methylpyrrolidin-3-yl)-4,6-dimethoxy-phenyl)prop-2-en-1-one

The title compound was obtained by reaction of compound of example 7 and 3-fluorobenzaldehyde (1.2 gm, 9.7 mmol) according to the procedure described in example 20, step 1.

Yield: 0.15 gm (36%); ¹H NMR (CDCl₃): δ 14.00 (s, 1H), 7.88 (d, 1H), 7.74 (d, 1H), 7.5-7.23 (m, 4H), 6.01 (s, 1H), 4.1 (m, 1H), 3.98 (s, 3H), 3.93 (s, 3H), 3.61 (dd, 1H), 3.42 (m, 1H), 3.16 (m, 1H), 2.78 (q, 1H), 2.62 (m, 1H), 2.39 (s, 3H), 2.01 (m, 2H); MS: m/e 416 (M+1).

### Example 36

### (+/−)3-(2-Fluorophenyl)-1-(2-hydroxy-3-(2-(hydroxymethyl)-1-methylpyrrolidin-3-yl)-4,6-dimethoxy-phenyl)prop-2-en-1-one

The title compound was obtained by reaction of compound of example 7 (1 gm, 3.2 mmol) and 2-fluorobenzaldehyde (1.2 gm, 9.7 mmol) according to the procedure described in example 20, step 1.

Yield: 0.13 gm, (31%); ¹H NMR (CDCl₃): δ 14.03 (s, 1H), 7.98 (d, 1H), 7.84 (d, 1H), 7.61-7.43 (m, 4H), 6.03 (s, 1H), 4.0 (m, 1H), 3.92 (s, 3H), 3.87 (s, 3H), 3.65 (dd, 1H), 3.32 (m, 1H), 3.18 (m, 1H), 2.40 (q, 1H), 2.62 (m, 1H), 2.36 (s, 3H), 1.97 (m, 2H); MS: m/e 416 (M+1).

### Example 37

### (+/−)1-(2-Hydroxy-3-(2-(hydroxymethyl)-1-methylpyrrolidin-3-yl)-4,6-dimethoxy-phenyl)-3-(3-hydroxy-4-methoxyphenyl)prop-2-en-1-one

The title compound was obtained by reaction of compound of example 7 (1 gm, 3.2 mmol) and 3-hydroxy-4-methoxybenzaldehyde (1.47 gm, 9.7 mmol) according to the procedure described in example 20, step 1.

Yield: 0.18 gm, (40%); ¹H NMR (CDCl₃): δ 14.1 (s, 1H), 7.95 (d, 1H), 7.89 (d, 1H), 7.51-7.41 (m, 3H), 6.01 (s, 1H), 4.03 (m, 1H), 3.95 (s, 3H), 3.98 (s, 3H), 3.86 (s, 3H), 3.61 (dd, 1H), 3.32 (m, 1H), 3.18 (m, 1H), 2.38 (q, 1H), 2.59 (m, 1H), 2.36 (s, 3H), 2.04 (m, 2H); MS: m/e 444 (M+1).

### Example 38

### (+/−)3-(2-Chlorophenyl)-1-(2-hydroxy-3-(4-(hydroxymethyl)-1-methylpyrrolidin-3-yl)-4,6-dimethoxy-phenyl)prop-2-en-1-one

The title compound was obtained by reaction of compound of example 15 (1 gm, 3.2 mmol) and 2-chlorobenzaldehyde (1.36 gm, 9.7 mmol) according to the procedure described in example 20, step 1.

Yield: 0.68 gm (49.27%); ¹HNMR (CDCl₃): δ 13.8 (s, 1H), 7.99 (d, 1H), 7.94 (d, 1H), 7.67 (m, 1H), 7.4 (m, 1H), 7.28 (m, 2H), 6.0 (s, 1H), 3.95 (s, 3H), 3.88 (s, 3H), 3.8 (m, 1H), 3.64 (m, 2H), 3.09 (m, 1H), 2.86 (m, 1H), 2.71 (m, 1H), 2.52 (m, 1H), 2.39 (s, 3H), 2.17 (m, 1H); MS: m/e 432 (M+1).

### Example 39

### (+/−)4-(3-(3-(2-Chlorophenyl)acryloyl)-2-hydroxy-4,6-dimethoxy-phenyl)-3-(hydroxymethyl)-1-methylpyrrolidin-2-one

The title compound was obtained by reaction of compound of example 19 (1 gm, 3.2 mmol) and 2-chlorobenzaldehyde (1.36 gm, 9.7 mmol) according to the procedure described in example 20, step 1.

Yield: 0.68 gm (49.27%); ¹HNMR (CDCl₃): δ 14.28 (s, 1H), 8.17 (d, 1H), 7.85 (d, 1H), 7.7 (m, 1H), 7.45 (m, 1H), 7.32 (m, 2H), 6.01 (s, 1H), 3.96 (s, 3H), 3.91 (s, 3H), 3.87 (m, 1H), 3.73 (m, 3H), 3.38 (m, 1H), 3.21 (m, 1H), 2.91 (s, 3H); MS: m/e 446 (M+1).

### Example 40

### 2,4,6-Trimethoxy-benzonitrile

To a mixture of compound of example 8 (40 gm, 0.204×10³ mmole) and iodine (56.96 gm, 0.225×10³ mmole) in ammonia water (1.6 L of 28% solution) and THF (400 ml) stirred at room temperature for 1 hour, was added an aq. solution of sodium thiosulphate (Na₂S₂O₃). The precipitate obtained was filtered, washed with water, and recrystallized using EtOAc to obtain the title compound.

Yield: 38 gm (95%); ¹H NMR (CDCl₃): δ 6.15 (s, 2H), 4.0 (s, 6H), 3.85 (s, 3H); MS: m/e 194 (M+1).

### Example 41

### 5-(2,4,6-Trimethoxy-phenyl)-2H tetrazole

To a mixture of compound of example 40 (25 gm, 0.129×10³ mmole) in water (250 ml) was added a mixture of sodium azide (8.84 gm, 0.136×10³ mmole) and zinc bromide (43.71 gm, 0.19×10³ mmole). The reaction mixture was refluxed for 24 hours with vigorous stirring, followed by addition of HCl (1.13 L, 1 M) and EtOAc (2 L). Stirring was continued until no solid was present and pH of the aqueous layer was 1. The organic layer was isolated and aqueous layer was extracted with EtOAc (600 ml×2). The combined organic layer was dried over Na₂SO₄ and evaporated under reduced pressure, followed by addition of 0.25 N NaOH (2.5 L). The mixture was stirred for 30 minutes. The suspension was filtered and washed with NaOH (130 ml). To the filtrate, was added 3N HCl (250 ml) with vigorous stirring to obtain a precipitate. The precipitate was filtered, washed with 3N HCl (130 ml×2) and dried in an oven to obtain the title compound.

Yield: 18.95 gm (62%); ¹H NMR (DMSO-d₆): δ 8.9 (s, 1H), 6.11 (s, 2H), 3.78-3.77 (two singlet, 9H); MS: m/e 237 (M+1).

### Example 42

### 2-Methyl-5-(2,4,6-trimethoxy-phenyl)-2H-tetrazole

To a solution of compound of example 41 (5 gm, 21.18×10³ mmole) in methanol (70 ml) was added formalin (1.5 gm, 56.17 mmole) and 10% palladium on charcoal under N₂ atmosphere. The reaction mixture was hydrogenated at 40 psi at room temperature for 3 hours. The reaction mixture was filtered through celite pad, and concentrated under reduced pressure. The crude product obtained was purified by column chromotography (silica gel, 5% MeOH and 1% liquor ammonia in chloroform) to obtain the title compound.

Yield: 4.93 gm (93%); ¹H NMR (DMSO-d₆): δ 6.11 (s, 2H), 3.80 (s, 6H), 3.78 (s, 3H); MS: m/e 251 (M+1).

### Example 43

### 1-[2-Hydroxy-4,6-dimethoxy-3-(2-methyl-2H tetrazole-5-yl)-phenyl]ethanone

To a solution of compound of example 42 (5 gm, 20 mmol) in acetic anhydride (8.5 ml, 0.089×10³ mmole) was added boron trifluoride etherate (10.02 ml, 0.079×10³ mmol) with stirring at 0° C. under N₂ atmosphere. The reaction mixture was stirred at room temperature for 4 hours, poured over crushed ice (0.25 kg) and made alkaline by adding saturated aq. Na₂CO₃ solution. The reaction mixture was extracted using CHCl₃ (150 ml×3), and the organic layer was washed with brine, dried over anhydrous Na₂SO₄ and concentrated. The crude product obtained was purified by column chromatography (silica gel, 5% MeOH and 1% liquor ammonia in chloroform) to obtain the title compound.

Yield: 3.08 gm (55.39%); ¹H NMR (DMSO-d₆): δ 6.11 (s, 2H), 3.80 (s, 6H), 3.78 (s, 3H); MS: m/e 279 (M+1).

### Example 44

### (+/−)3-(3-Bromophenyl)-1-(2-hydroxy-4,6-dimethoxy-3-(2-methyl-2H tetrazol-5-yl)phenyl)prop-2-en-1-one

The title compound was obtained by reaction of compound of example 43 (1 gm, 3.597×10³ mmole) and 3-bromobenzaldehyde (2 gm, 10.79 mmol) according to the procedure described in example 20, step 1.

Yield: 0.783 gm (49%); ¹H NMR (DMSO-d₆): δ 14.00 (s, 1H), 7.8 (d, 1H), 7.76 (d, 1H), 7.44 (s, 1H), 7.39 (d, 2H), 7.25 (t, 1H), 6.09 (s, 1H), 3.84 (s, 3H), 3.89 (s, 3H), 3.63 (s, 3H); MS: m/e 445 (M+1).

### Example 45

### (+/−)1-(2-Hydroxy-3-(2-(hydroxymethyl)-1-methylpyrrolidin-3-yl)-4,6-dimethoxy-phenyl)-3-(5-methylfuran-2-yl)prop-2-en-1-one

The title compound was obtained by reaction of compound of example 7 (1 gm, 3.2 mmol) and 5-methylfurfural (0.97 ml, 9.7 mmol) according to the procedure described in example 20, step 1.

Yield: 0.66 gm (51%); ¹HNMR (CDCl₃): δ 7.69 (d, 1H), 7.55 (d, 1H), 6.58 (d, 1H), 6.11 (d, 1H), 5.99 (s, 1H), 3.95 (s, 3H), 3.92 (m, 1H), 3.89 (s, 3H), 3.62 (dd, 1H), 3.41 (m, 1H), 3.15 (m, 1H), 2.79 (m, 1H), 2.6 (m, 1H), 2.38 (s, 3H), 2.02 (m, 2H); MS: m/e 402 (M+1).

### Example 46

### (+/−)3-(5-Bromofuran-2-yl)-1-(2-hydroxy-3-(2-(hydroxymethyl)-1-methylpyrrolidin-3-yl)-4,6-dimethoxy-phenyl)prop-2-en-1-one

The title compound was obtained by reaction of compound of example 7 (1 gm, 3.2 mmol) and 5-bromo-2-furaldehyde (1.7 gm, 9.7 mmol) according to the procedure described in example 20, step 1.

Yield; 0.88 gm (59%); ¹HNMR (CDCl₃): δ 7.75 (d, 1H), 7.47 (d, 1H), 6.6 (d, 1H), 6.43 (d, 1H), 6.0 (s, 1H), 3.92 (s, 3H), 3.90 (m, 1H), 3.88 (s, 3H), 3.61 (dd, 1H), 3.41 (m, 1H), 3.17 (m, 1H), 2.79 (m, 1H), 2.61 (m, 1H), 2.36 (s, 3H), 2.02 (m, 2H); MS: m/e 466, 468 (M+1).

### Example 47

### (+/−)3-(4-Bromothiophen-2-yl)-1-(2-hydroxy-3-(2-(hydroxymethyl)-1-methyl pyrrolidin-3-yl)-4,6-dimethoxy-phenyl)prop-2-en-1-one

The title compound was obtained by reaction of compound of example 7 (1 gm, 3.2 mmol) and 4-bromo-2-thiophenecarboxaldehyde (1.85 gm, 9.7 mmol) according to the procedure described in example 20, step 1.

Yield: 0.88 gm (57%); ¹HNMR (CDCl₃): δ 7.80 (d, 1H), 7.69 (d, 1H), 7.25 (s, 1H), 7.19 (s, 1H), 5.99 (s, 1H), 3.92 (s, 3H), 3.90 (m, 1H), 3.89 (s, 3H), 3.61 (dd, 1H), 3.39 (m, 1H), 3.15 (m, 1H), 2.79 (m, 1H), 2.61 (m, 1H), 2.36 (s, 3H), 2.02 (m, 2H); MS: m/e 482, 484 (M+1).

### Example 48

### (+/−)1-(2-Hydroxy-3-(2-(hydroxymethyl)-1-methylpyrrolidin-3-yl)-4,6-dimethoxy-phenyl)-3-(pyridin-3-yl)prop-2-en-1-one

The title compound was obtained by reaction of compound of example 7 (1 gm, 3.2 mmol) and 3-pyridinecarboxaldehyde (0.91 ml, 9.7 mmol) according to the procedure described in example 20, step 1.

Yield: 0.68 gm (48.1%); ¹HNMR (CDCl₃): δ 8.84 (s, 1H), 8.6 (d, 1H), 7.92 (d, 1H), 7.87 (d, 1H), 7.73 (d, 1H), 7.36 (m, 1H), 6.01 (s, 1H), 3.99 (s, 3H), 3.96 (s, 3H), 3.94 (m, 1H), 3.63 (dd, 1H), 3.40 (m, 1H), 3.16 (m, 1H), 2.79 (m, 1H), 2.6 (m, 1H), 2.36 (s, 3H), 2.02 (m, 2H); MS: m/e 399 (M+1).

### Example 49

### (+/−)-1-[2-Hydroxy-3-(2-hydroxymethyl-1-methyl-pyrrolidin-3-yl)-4,6-dimethoxy-phenyl]-3-(3-trifluoromethyl-phenyl)-prop-2-en-1-one

The title compound was obtained by reaction of compound of example 7 (1 gm, 3.2 mmol) and 3-trifluoromethyl-benzaldehyde (1.56 g, 9.6 mmol) according to the procedure described in example 20, step 1.

Yield: 40%; ¹H NMR (CDCl₃): δ 7.80 (d, 1H, J=15.3), 7.76 (s, 1H), 7.70 (d, 1H, J=15.3), 7.48 (d, 2H), 7.26 (m, 1H), 6.0 (s, 1H), 3.98 (m, 1H), 3.96 (s, 3H), 3.90 (s, 3H), 3.58 (dd, 1H), 3.40 (m, 1H), 3.16 (m, 1H), 2.76 (m, 1H), 2.58 (m, 1H), 2.36 (s, 3H), 2.0 (m, 2H); MS: m/e 466 (M+1).

### Pharmacological Data

The efficacy of the present compounds can be determined by a number of pharmacological assays well known in the art, such as described below. The exemplified pharmacological assays, which follow herein below, have been carried out with the compounds of the present invention.

### Example 50

### Primary Screening In Vitro—Whole Blood Cell Culture Assay

TNF-α production by lipopolysaccharide (LPS) in whole blood was measured according to the method described in literature (J. Immunol. Methods, 1991, 139, 233-240).

Blood was collected from healthy donors into potassium EDTA vacutainer tubes (Becton Dickinson) and diluted with RPMI (Roswell Park Memorial Institute) 1640 culture medium (Gibco BRL, Pasley, UK) containing 100 U/ml penicillin and 100 μg/ml streptomycin, (100× solution, Sigma Chemical Co. St Louis, Mo.) with no added serum. The white blood cell count was adjusted to 1×10⁶ cells/ml and 100 it/well of the diluted blood was transferred into 96-well culture plates. Following cell plating, 79 μl of culture medium and 1 μl of the test compounds (final concentration 1 μM and 10 μM) dissolved in DMSO was added to the cells. The final concentration of DMSO was adjusted to 0.5%. 1 μl of vehicle (0.5% DMSO) was used as control. Rolipram (300 μM) was used as a standard compound. The plates were incubated for 30 minutes at 37° C. in an atmosphere of 5% CO₂. Finally, 20 μl (10 μg/ml) per well of LPS (Escherchia coli 0127:B8, Sigma Chemical Co. St. Louis, Mo.) was added, for a final concentration of 1 μg/ml. Plates were incubated at 37° C. for 4.5 hours in an atmosphere of 5% CO₂. Supernatants were harvested and assayed for TNF-α by ELISA as described by the manufacturer (OptiEIA ELISA sets, BD Biosciences, Pharmingen). Percent inhibition of TNF-α release in comparison to the control was calculated.

The results of representative compounds of the present invention are summarized in Table 1.

TABLE 1
% inhibition of TNF-α release in whole blood cell culture assay

| Example | % inhibition of TNF-α release |
|---|---|
| No. | (at a concentration of 10 μM) |
| 20 | 66 |
| 21 | 61 |
| 23 | 32 |
| 25 | 68 |
| 28 | 30 |
| 29 | 31 |
| 39 | 75 |
| 45 | 54 |
| 48 | 61 |

Conclusion: Representative compounds of the present invention are found to inhibit TNF-α release.

### Example 51

### Secondary Screening In Vitro—Human Peripheral Blood Mononuclear Cells (hPBMCs)

TNF-α production by lipopolysaccharides (LPS) in hPBMCs was measured according to the method described in literature (Physiol. Res., 2003, 52, 593-598). Blood was collected from healthy donors into Potassium EDTA vacutainer tubes (BD vacutainer). The PBMCs were isolated using gradient centrifugation in Histopaque-1077 solution (Sigma). Isolated PBMC were suspended in RPMI 1640 culture medium (Sigma-Aldrich Fine Chemicals, USA), containing 10% fetal bovine serum (FBS) (JRH, USA), 100 U/ml penicillin (Sigma Chemical Co. St Louis, Mo.) and 100 μg/ml streptomycin (Sigma Chemical Co. St Louis, Mo.). The cell concentration was adjusted to 1×10⁶ cells/ml. The viability as determined by trypan blue dye exclusion was uniformly ≧98%. The cell suspension (100 μl) was added to the wells of a 96-well culture plate. Following cell plating, 79 μl of the culture medium and 1 μl of eight different concentrations of the test compounds (final concentration 0.03, 0.1, 0.3, 1, 3, 10, 30, 100 μg/ml) dissolved in DMSO were added to the cells. The final concentration of DMSO was adjusted to 0.5%. The vehicle (0.5 DMSO) was used as control. Rolipram (300 μM) was used as a standard compound. The plates were incubated for 30 minutes at 37° C. in an atmosphere of 5% CO₂. Finally, 20 μl (10 μg/ml) per well of LPS, (Escherchia coli 0127:B8, Sigma Chemical Co., St. Louis, Mo.) was added, for a final concentration of 1 □g/ml. The plates were incubated at 37° C. for 5 hours in an atmosphere of 5% CO₂. To assess the cytotoxic effect of the test compounds, the cellular viability test was performed using MTS (3-(4,5-dimethylthiazol-2-yl)-5-(3-carboxymethoxyphenyl)-2-(4-sulfonyl)-2H-tetrazolium) reagent after 5 hours of incubation. Supernatants were harvested and assayed for TNF-α, interleukin-1β (IL-1β), interleukin-6 (IL-6) and interleukin-8 (IL-8) by ELISA as described by the manufacturer. (OptiEIA ELISA sets, BD Biosciences, Pharmingen). The 50% inhibitory concentration (IC₅₀) values were calculated by a nonlinear regression method using GraphPad software (Prism 3.03) The results of representative compounds of the present invention are summarized in Table 2

TABLE 2
IC₅₀ (μM) values in human peripheral blood mononuclear cells
(hPBMCs) assay

IC₅₀ (μM)

| Example No. | TNF-α | IL-1β | IL-6 | IL-8 |
|---|---|---|---|---|
| 20 | 0.9 | 0.8 | 4.9 | 6.5 |
| 21 | 0.8 | 0.2 | 2.3 | 5.2 |
| 25 | 0.5 | 0.1 | 2.5 | 5.4 |

Conclusion: Representative compounds of the present invention are found to be active in human peripheral blood mononuclear cells (hPBMCs) assay.

It should be noted that, as used in this specification and the appended claims, the singular forms “a,” “an,” and “the” include plural referents unless the content clearly dictates otherwise. Thus, for example, reference to a composition containing “a compound” includes a mixture of two or more compounds. It should also be noted that the term “or” is generally employed in its sense including “and/or” unless the content clearly dictates otherwise.

All publications and patent applications in this specification are indicative of the level of ordinary skill in the art to which this invention pertains.

The invention has been described with reference to various specific and preferred embodiments and techniques. However, it should be understood that many variations and modifications may be made while remaining within the spirit and scope of the invention.

1. A compound of formula (I): 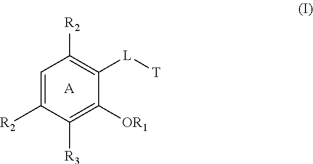
wherein,
R₁ is selected from hydrogen, alkyl or —C(O)-alkyl;
R₂ at each occurrence is independently selected from hydrogen, halogen, hydroxy, alkyl, alkoxy
or —O—C—(O)-alkyl;
R₃ is selected from the groups of formula (i) to (iv) 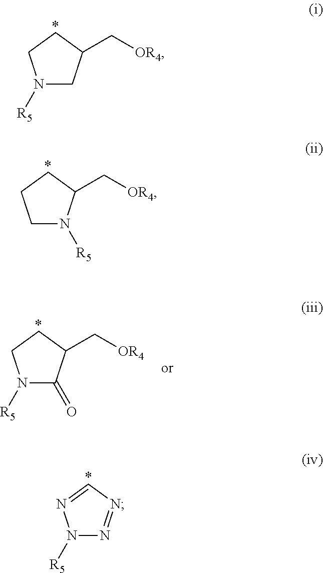
* indicates the point of attachment;
R₄ is selected from hydrogen, alkyl or —C(O)-alkyl;
R₅ is selected from hydrogen or alkyl;
L is selected from the groups of formula: 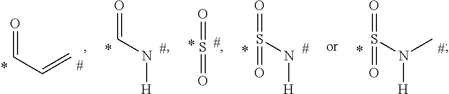
* indicates the point of attachment to phenyl ring A;
# indicates the point of attachment to T; and
T is selected from phenyl or 5 or 6 membered heteroaryl; wherein the phenyl and heteroaryl are unsubstituted or substituted by at least one group selected from halogen, hydroxy, alkyl, haloalkyl, alkoxy, carboxy, amino, nitro or cyano;
or a stereoisomer, a tautomer or a pharmaceutically acceptable salt thereof.

2. The compound of formula (I) according to claim 1, wherein
R₁ is selected from hydrogen or alkyl;
R₂ at each occurrence is independently selected from hydrogen, halogen, hydroxy, alkyl or alkoxy; and
T is phenyl; which is unsubstituted or substituted by at least one group selected from halogen, hydroxy, alkyl, haloalkyl, alkoxy, carboxy, amino, nitro or cyano;
or a stereoisomer, a tautomer or a pharmaceutically acceptable salt thereof.

3. The compound of formula (I) according to claim 1, wherein
R₁ is hydrogen;
R₂ is alkoxy;
R₃ is the group of formula (ii) 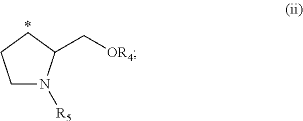
* indicates the point of attachment;
R₄ is selected from hydrogen or —C(O)-alkyl;
R₅ is alkyl; and
T is phenyl; which is unsubstituted or substituted by at least one group selected from halogen, hydroxy, alkyl, haloalkyl, alkoxy, carboxy, amino, nitro or cyano; or a stereoisomer, a tautomer or a pharmaceutically acceptable salt thereof.

4. The compound of formula (I) according to claim 1, wherein
R₁ is hydrogen;
R₂ is alkoxy;
R₃ is the group of formula (ii) 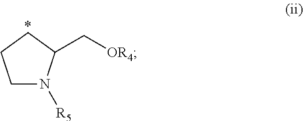
* indicates the point of attachment;
R₄ is selected from hydrogen or —C(O)-alkyl;
R₅ is alkyl;
L is 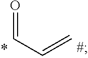
* indicates the point of attachment to phenyl ring A;
# indicates the point of attachment to T; and
T is phenyl; which is unsubstituted or substituted by at least one group selected from halogen, hydroxy, alkyl, haloalkyl, alkoxy, carboxy, amino, nitro or cyano; or a stereoisomer, a tautomer or a pharmaceutically acceptable salt thereof.

5. The compound of formula (I) according to claim 1, wherein
R₁ is hydrogen;
R₂ is alkoxy;
R₃ is selected from the groups of formula (i), (iii) or (iv); 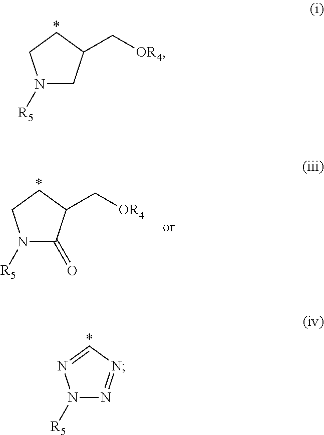
* indicates the point of attachment;
R₄ is hydrogen;
R₅ is alkyl;
L is 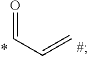
* indicates the point of attachment to phenyl ring A;
# indicates the point of attachment to T; and
T is phenyl; which is unsubstituted or substituted by at least one group selected from halogen, hydroxy, alkyl, haloalkyl, alkoxy, carboxy, amino, nitro or cyano; or a stereoisomer, a tautomer or a pharmaceutically acceptable salt thereof.

6. The compound of formula (I) according to claim 1, wherein
R₁ is selected from hydrogen or alkyl;
R₂ at each occurrence is independently selected from hydrogen, halogen, hydroxy, alkyl or alkoxy; and
T is 5 or 6 membered heteroaryl; wherein the heteroaryl is unsubstituted or substituted by at least one group selected from; halogen, hydroxy, alkyl, haloalkyl, alkoxy, carboxy, amino, nitro or cyano;
or a stereoisomer, a tautomer or a pharmaceutically acceptable salt thereof.

7. The compound of formula (I) according to claim 1, wherein
R₁ is hydrogen;
R₂ is alkoxy;
R₃ is the group of formula (ii) 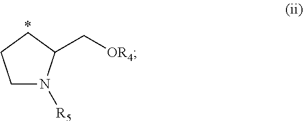
* indicates the point of attachment;
R₄ is hydrogen;
R₅ is alkyl; and
T is 5 membered heteroaryl selected from furanyl, thiophenyl, imidazolyl, oxazolyl, isoxazolyl or thiazolyl; wherein the heteroaryl is unsubstituted or substituted by at least one group selected halogen, hydroxy, alkyl, haloalkyl, alkoxy, carboxy, amino, nitro or cyano; or a stereoisomer, a tautomer or a pharmaceutically acceptable salt thereof.

8. The compound of formula (I) according to claim 1, wherein
R₁ is hydrogen;
R₂ is alkoxy;
R₃ is the group of formula (ii) 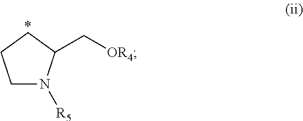
* indicates the point of attachment;
R₄ is hydrogen;
R₅ is alkyl;
L is 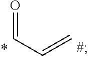
* indicates the point of attachment to phenyl ring A;
# indicates the point of attachment to T; and
T is 5 membered heteroaryl selected from furanyl, thiophenyl, imidazolyl, oxazolyl, isoxazolyl or thiazolyl; wherein the heteroaryl is unsubstituted or substituted by at least one group selected from halogen, hydroxy, alkyl, haloalkyl, alkoxy, carboxy, amino, nitro or cyano; or a stereoisomer, a tautomer or a pharmaceutically acceptable salt thereof.

9. The compound of formula (I) according to claim 1, wherein
R₁ is hydrogen;
R₂ is alkoxy;
R₃ is a group of formula (ii) 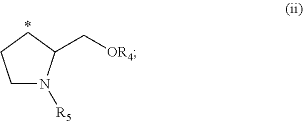
* indicates the point of attachment;
R₄ is hydrogen;
R₅ is alkyl;
L is 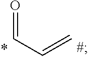
* indicates the point of attachment to phenyl ring A;
# indicates the point of attachment to T; and
T is selected from furanyl or thiophenyl; wherein the furanyl or thiophenyl are unsubstituted or substituted by at least one group selected from halogen or alkyl; or a stereoisomer, a tautomer or a pharmaceutically acceptable salt thereof.

10. The compound of formula (I) according to claim 1, wherein
R₁ is hydrogen;
R₂ is alkoxy;
R₃ is a group of formula (ii) 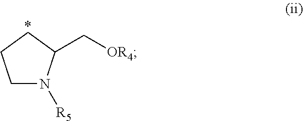
* indicates the point of attachment;
R₄ is hydrogen;
R₅ is alkyl; and
T is 6 membered heteroaryl selected from pyrazinyl, pyridinyl, pyrimidinyl, or pyridazinyl; wherein the heteroaryl is unsubstituted or substituted by at least one group selected from halogen, hydroxy, alkyl, haloalkyl, alkoxy, carboxy, amino, nitro or cyano; or a stereoisomer, a tautomer or a pharmaceutically acceptable salt thereof.

11. The compound of formula (I) according to claim 1, wherein
R₁ is hydrogen;
R₂ is alkoxy;
R₃ is the group of formula (ii) 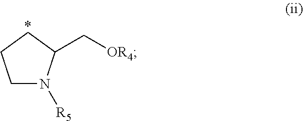
* indicates the point of attachment;
R₄ is hydrogen;
R₅ is alkyl;
L is 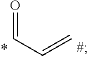
* indicates the point of attachment to phenyl ring A;
# indicates the point of attachment to T; and
T is 6 membered heteroaryl selected from pyrazinyl, pyridinyl, pyrimidinyl, or pyridazinyl; wherein the heteroaryl ring is unsubstituted or substituted by at least one group selected from halogen, hydroxy, alkyl, haloalkyl, alkoxy, carboxy, amino, nitro or cyano; or a stereoisomer, a tautomer or a pharmaceutically acceptable salt thereof.

12. The compound of formula (I) according to claim 1, wherein
R₁ is hydrogen;
R₂ is alkoxy;
R₃ is the group of formula (ii) 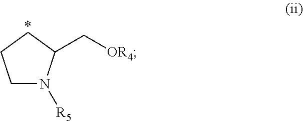
* indicates the point of attachment;
R₄ is hydrogen;
R₅ is alkyl;
L is 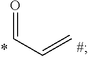
* indicates the point of attachment to phenyl ring A;
# indicates the point of attachment to T; and
T is pyridinyl; wherein the pyridinyl is unsubstituted or substituted by at least group selected from halogen or alkyl; or a stereoisomer, a tautomer or a pharmaceutically acceptable salt thereof.

13. The compound of formula (I) according to claim 1, wherein
R₁ is hydrogen;
R₂ is alkoxy;
R₃ is selected from groups of formula (i), (iii) or (iv); 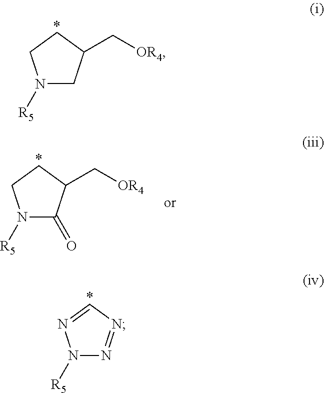
* indicates the point of attachment;
R₄ is hydrogen;
R₅ is alkyl;
L is 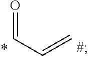
* indicates the point of attachment to phenyl ring A;
# indicates the point of attachment to T; and
T is 5 or 6 membered heteroaryl; wherein the heteroaryl is unsubstituted or substituted by at least one group selected from halogen, hydroxy, alkyl, haloalkyl, alkoxy, carboxy, amino, nitro or cyano; or a stereoisomer, a tautomer or a pharmaceutically acceptable salt thereof.

14. The compound of formula (I) according to claim 1, wherein the compound is:
(+/−)3-(2-Chlorophenyl)-1-(2-hydroxy-3-(2-(hydroxymethyl)-1-methylpyrrolidin-3-yl)-4,6-dimethoxy-phenyl)prop-2-en-1-one hydrochloride,
(−)3-(2-Chlorophenyl)-1-(2-hydroxy-3-(2-(hydroxymethyl)-1-methylpyrrolidin-3-yl)-4,6-dimethoxy-phenyl)prop-2-en-1-one,
(+)3-(2-Chlorophenyl)-1-(2-hydroxy-3-(2-(hydroxymethyl)-1-methylpyrrolidin-3-yl)-4,6-dimethoxy-phenyl)prop-2-en-1-one,
(+/−)3-(3-Bromophenyl)-1-(2-hydroxy-3-(2-(hydroxymethyl)-1-methylpyrrolidin-3-yl)-4,6-dimethoxy-phenyl)prop-2-en-1-one,
(+/−)3-(2,4-Dimethoxy-phenyl)-1-(2-hydroxy-3-(2-(hydroxymethyl)-1-methylpyrrolidin-3-yl)-4,6-dimethoxy-phenyl)prop-2-en-1-one,
(+/−)1-(2-Hydroxy-3-(2-(hydroxymethyl)-1-methylpyrrolidin-3-yl)-4,6-dimethoxyphenyl)-3-m-tolylprop-2-en-1-one,
(+/−)1-(2-Hydroxy-3-(2-(hydroxymethyl)-1-methylpyrrolidin-3-yl)-4,6-dimethoxy-phenyl)-3-o-tolylprop-2-en-1-one,
(+/−)1-(2-Hydroxy-3-(2-(hydroxymethyl)-1-methylpyrrolidin-3-yl)-4,6-dimethoxy-phenyl)-3-(3-nitrophenyl)prop-2-en-1-one,
(+/−)1-(2-Hydroxy-3-(2-(hydroxymethyl)-1-methylpyrrolidin-3-yl)-4,6-dimethoxy-phenyl)-3-(4-nitrophenyl)prop-2-en-1-one,
(+/−)3-(2-Bromophenyl)-1-(2-hydroxy-3-(2-(hydroxymethyl)-1-methylpyrrolidin-3-yl)-4,6-dimethoxy-phenyl)prop-2-en-1-one,
(+/−)3-(4-Chlorophenyl)-1-(2-hydroxy-3-(2-(hydroxymethyl)-1-methylpyrrolidin-3-yl)-4,6-dimethoxy-phenyl)prop-2-en-1-one,
(+/−)3-(4-Fluorophenyl)-1-(2-hydroxy-3-(2-(hydroxymethyl)-1-methylpyrrolidin-3-yl)-4,6-dimethoxyphenyl)prop-2-en-1-one,
(+/−)1-(2-Hydroxy-3-(2-(hydroxymethyl)-1-methylpyrrolidin-3-yl)-4,6-dimethoxy-phenyl)-3-(4-methoxyphenyl)prop-2-en-1-one,
(+/−)1-(2-Hydroxy-3-(2-(hydroxymethyl)-1-methylpyrrolidin-3-yl)-4,6-dimethoxy-phenyl)-3-p-tolylprop-2-en-1-one,
(+/−)1-(2-Hydroxy-3-(2-(hydroxymethyl)-1-methylpyrrolidin-3-yl)-4,6-dimethoxy-phenyl)-3-phenylprop-2-en-1-one,
(+/−)3-(3-Aminophenyl)-1-(2-hydroxy-3-(2-(hydroxymethyl)-1-methylpyrrolidin-3-yl)-4,6-dimethoxy-phenyl)prop-2-en-1-one,
(+/−)Acetic acid (3-(3-(3-(2-chlorophenyl)acryloyl)-2-hydroxy-4,6-dimethoxy-phenyl)-1-methylpyrrolidin-2-ylmethyl ester,
(+/−)3-(3-Fluorophenyl)-1-(2-hydroxy-3-(2-(hydroxymethyl)-1-methylpyrrolidin-3-yl)-4,6-dimethoxy-phenyl)prop-2-en-1-one,
(+/−)3-(2-Fluorophenyl)-1-(2-hydroxy-3-(2-(hydroxymethyl)-1-methylpyrrolidin-3-yl)-4,6-dimethoxy-phenyl)prop-2-en-1-one,
(+/−)1-(2-Hydroxy-3-(2-(hydroxymethyl)-1-methylpyrrolidin-3-yl)-4,6-dimethoxyphenyl)-3-(3-hydroxy-4-methoxyphenyl)prop-2-en-1-one,
(+/−)3-(2-Chlorophenyl)-1-(2-hydroxy-3-(4-(hydroxymethyl)-1-methylpyrrolidin-3-yl)-4,6-dimethoxy-phenyl)prop-2-en-1-one,
(+/−)4-(3-(3-(2-Chlorophenyl)acryloyl)-2-hydroxy-4,6-dimethoxy-phenyl)-3-(hydroxymethyl)-1-methylpyrrolidin-2-one,
(+/−)3-(3-Bromophenyl)-1-(2-hydroxy-4,6-dimethoxy-3-(2-methyl-2H tetrazol-5-yl) phenyl)prop-2-en-1-one,
(1-(2-Hydroxy-3-(2-(hydroxymethyl)-1-methylpyrrolidin-3-yl)-4,6-dimethoxy-phenyl)-3-(4-methylfuran-2-yl)prop-2-en-1-one,
(+/−)3-(5-Bromofuran-2-yl)-1-(2-hydroxy-3-(2-(hydroxymethyl)-1-methylpyrrolidin-3-yl)-4,6-dimethoxy-phenyl)prop-2-en-1-one,
(+/−)3-(4-Bromothiophen-2-yl)-1-(2-hydroxy-3-(2-(hydroxymethyl)-1-methylpyrrolidin-3-yl)-4,6-dimethoxy-phenyl)prop-2-en-1-one,
(+/−)1-(2-Hydroxy-3-(2-(hydroxymethyl)-1-methylpyrrolidin-3-yl)-4,6-dimethoxy-phenyl)-3-(pyridin-3-yl)prop-2-en-1-one, or
(+/−)-1-[2-Hydroxy-3-(2-hydroxymethyl-1-methyl-pyrrolidin-3-yl)-4,6-dimethoxy-phenyl]-3-(3-trifluoromethyl-phenyl)prop-2-en-1-one,
or a pharmaceutically acceptable salt thereof.

15. A process for the preparation of the compound of formula (I), 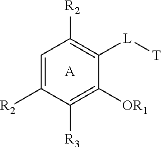
wherein,
R₁ is hydrogen; R₂ is hydroxy or alkoxy;
R₃ is a group of formula (ii), 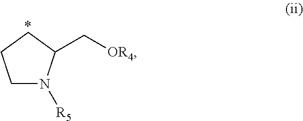
* indicates the point of attachment;
R₄ is hydrogen; R₅ is hydrogen or alkyl;
L is 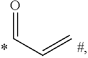
* indicates the point of attachment to phenyl ring A;
# indicates the point of attachment to T; wherein T is selected from phenyl or 5 or 6 membered heteroaryl; wherein the phenyl and heteroaryl are unsubstituted or substituted by at least one group selected from halogen, hydroxy, alkyl, haloalkyl, alkoxy, carboxy, amino, nitro or cyano), which process comprises:
step 1) reacting compound of formula (1a) (wherein R₁ is hydrogen; R₂ is hydroxy or alkoxy; R₄ is hydrogen; and R₅ is hydrogen or alkyl), 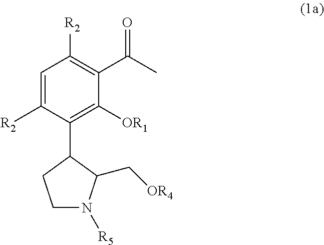
with a compound of formula T-CHO (wherein T is as defined above), in the presence of an aqueous alcoholic alkali; wherein the alkali is selected from sodium hydroxide or potassium hydroxide; to obtain the compound of formula (I); and
step 2) optionally converting the resulting compound of formula (I), to its corresponding pharmaceutically acceptable salt.

16. A process for the preparation of the compound of formula (I), 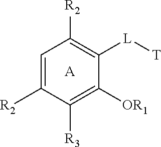
wherein,
R₁ is hydrogen; R₂ is hydroxy or alkoxy; R₃ is a group of formula (i), 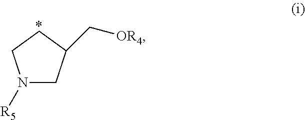
* indicates the point of attachment;
R₄ is hydrogen; R₅ is methyl; L is 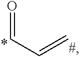
* indicates the point of attachment to phenyl ring A;
# indicates the point of attachment to T; wherein T is selected from phenyl or 5 or 6 membered heteroaryl; wherein the phenyl and heteroaryl are unsubstituted or substituted by at least one group selected from halogen, hydroxy, alkyl, haloalkyl, alkoxy, carboxy, amino, nitro or cyano, which process comprises:
step 1) reacting a compound of formula (2d) (wherein R₁ is alkyl; R₂ is hydroxy or alkoxy; and R₅ is hydrogen), 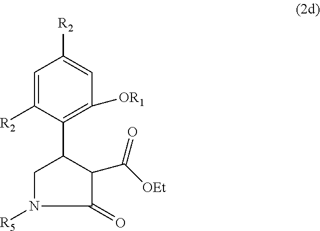
with a reagent selected from sodium cyanoborohydride, sodium triacetoxy borohydride, lithium aluminum hydride, borane in tetrahydrofuran or borane dimethyl sulfide, in a solvent selected from diethyl ether, tetrahydrofuran or dioxane at reflux temperature to obtain compound of formula (2e) (wherein R₁ is alkyl; R₂ is hydroxy or alkoxy; and R₅ is hydrogen); 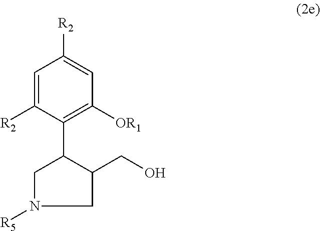
step 2) reacting the compound of formula (2e) with 10% palladium on charcoal in presence of formalin; in methanol at a temperature in the range of 20-55° C. and pressure in the range of 40-60 psi; to obtain compound of formula (20 (wherein R₁ is alkyl; R₂ is hydroxy or alkoxy; and R₅ is methyl); 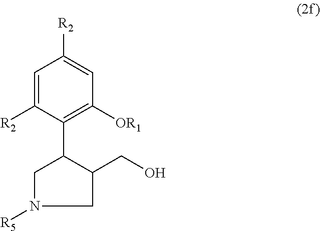
step 3) reacting the compound of formula (2f) with an acylating agent in the presence of a Lewis acid and a solvent at a temperature in the range of 0° C. to 40° C.; wherein the acylating agent is selected from acetic anhydride and acetyl chloride; the Lewis acid is selected from aluminium chloride (AlCl₃), zinc chloride (ZnCl₂), zinc bromide (ZnBr₂) or boron trifluoride etherate; the solvent is a chlorinated solvent selected from dichloromethane or chloroform; to obtain compound of formula (2g) (wherein R₁ is hydrogen; R₂ is hydroxy or alkoxy; and R₅ is methyl); 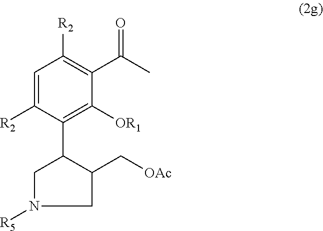
step 4) deacylation of the compound of formula (2g) using alkali hydroxide in water or in alcohol selected from methanol or ethanol; at a temperature in the range of 10° C. to reflux temperature; wherein the alkali hydroxide is selected from lithium hydroxide, sodium hydroxide, barium hydroxide or potassium hydroxide to obtain compound of formula (2l) (wherein R₁ is hydrogen and R₅ is methyl), 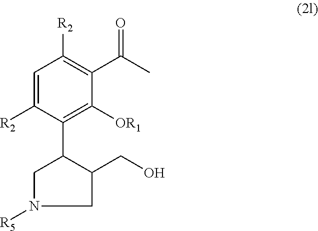
step 5) reacting the compound of formula (2l) with a compound of formula T-CHO (wherein T is as defined above) in the presence of an aqueous alcoholic alkali; wherein the alkali is selected from sodium hydroxide or potassium hydroxide; to obtain compound of formula (I); and
step 6) optionally converting the resulting compound of formula (I), to its corresponding pharmaceutically acceptable salt.

17. A process for the preparation of the compound of formula (I), 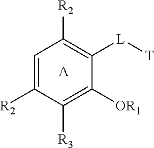
wherein,
R₁ is hydrogen; R₂ is, hydroxy or alkoxy; R₃ is a group of formula (iii), 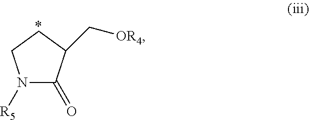
* indicates the point of attachment;
R₄ is hydrogen; R₅ is methyl; L is 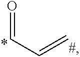
* indicates the point of attachment to phenyl ring A;
# indicates the point of attachment to T; wherein T is selected from phenyl or 5 or 6 membered heteroaryl; wherein the phenyl and heteroaryl are unsubstituted or substituted by at least one group selected from halogen, hydroxy, alkyl, haloalkyl, alkoxy, carboxy, amino, nitro or cyano), which process comprises:
step 1) reacting a compound of formula (2d) (wherein R₁ is alkyl; R₂ is hydroxy or alkoxy; and R₅ is hydrogen), 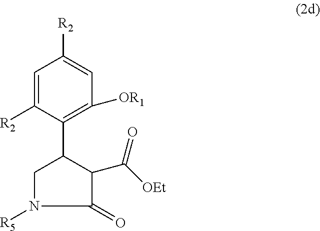
with an alkylating agent in presence of a base and a solvent at a temperature in the range of 0° C. to 40° C.; wherein the alkylating agent is selected from methyl iodide or dimethyl sulfate and the base is selected from sodium hydride or potassium tert-butoxide; the solvent is selected from diethyl ether, tetrahydrofuran, dioxane or aqueous alcohol; the alcohol is selected from methanol or ethanol to obtain compound of formula (2h) (wherein R₁ is alkyl; R₂ is hydroxy or alkoxy; and R₅ is methyl); 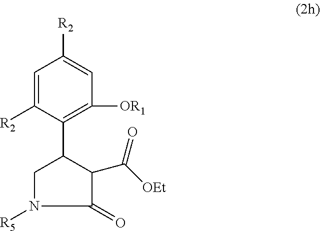
step 2) reducing the compound of formula (2h) using sodium borohydride in refluxing alcohol selected from methanol, ethanol, butanol or mixtures thereof, to obtain compound of formula (2i), (wherein R₁ is alkyl, R₂ is hydroxy or alkoxy; and R₅ is methyl); 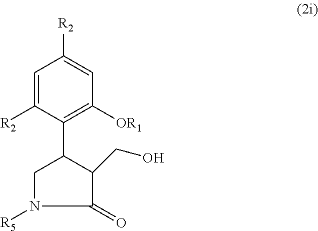
step 3) reacting the compound of formula (2i) with an acylating agent in the presence of a Lewis acid and a solvent at a temperature in the range of 0° C. to 40° C.; the acylating agent is selected from acetic anhydride and acetyl chloride; the Lewis acid is selected from aluminium chloride (AlCl₃), zinc chloride (ZnCl₂), zinc bromide (ZnBr₂) or boron trifluoride etherate; the solvent is a chlorinated solvent selected from dichloromethane or chloroform; to obtain compound of formula (2j) (wherein R₁ is hydrogen, R₂ is hydroxy or alkoxy; and R₅ are methyl); 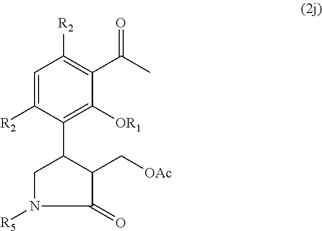
step 4) deacylation of the compound of formula (2j) using alkali hydroxide in water or in alcohol selected from methanol and ethanol; at a temperature in the range of 10° C. to reflux temperature; the alkali hydroxide is selected from lithium hydroxide, sodium hydroxide, barium hydroxide or potassium hydroxide; to obtain compounds of formula (2k) (wherein R₁ is hydrogen; R₂ is hydroxy or alkoxy; and R₅ is methyl); 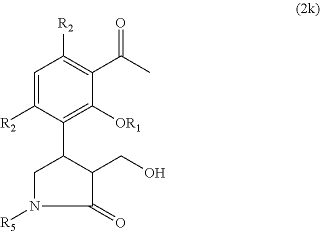
step 5) reacting the compound of formula (2k) with a compound of formula T-CHO (wherein T is as defined above) in the presence of an aqueous alcoholic alkali; wherein the alkali is selected from sodium hydroxide or potassium hydroxide; to obtain compound of formula (I); and
step 6) optionally converting the resulting compound of formula (I), to its corresponding pharmaceutically acceptable salt.

18. (canceled)

19. A pharmaceutical composition comprising a therapeutically effective amount of a compound of formula (I), according to claim 1, or a stereoisomer, a tautomer or a pharmaceutically acceptable salt, and a pharmaceutically acceptable carrier or a diluent.

20. A method for the treatment of a condition or a disorder mediated by one or more cytokines selected from Tumor necrosis factor alpha (TNF-α) or interleukins (IL-1, IL-6, IL-8), comprising administering to a mammal in need thereof a therapeutically effective amount of the compound of formula (I), according to claim 1, or a stereoisomer, a tautomer or a pharmaceutically acceptable salt thereof.

21. The method according to claim 20, wherein the condition or disorder is selected from inflammatory bowel disease, inflammation, rheumatoid arthritis, juvenile rheumatoid arthritis, psoriatic arthritis osteoarthritis, refractory rheumatoid arthritis, chronic non-rheumatoid arthritis, osteoporosis/bone resorption, Crohn's disease, septic shock, endotoxic shock, atherosclerosis, ischemia-reperfusion injury, coronary heart disease, vasculitis, amyloidosis, multiple sclerosis, sepsis, chronic recurrent uveitis, ulcerative colitis, cachexia, psoriasis, plasmocytoma, endometriosis, Behcet's disease, Wegener's granulomatosis, meningitis, autoimmune disease, immune deficiency, common variable immunodeficiency (CVID), chronic graft-versus-host disease, adult respiratory distress syndrome, pulmonary fibrosis, ankylosing spondylitis, systemic lupus erythematosus, allergic asthma or skin delayed type hypersensitivity.

22. The method according to claim 20, wherein the condition or disorder is selected from inflammatory bowel disease, inflammation, rheumatoid arthritis, juvenile rheumatoid arthritis, psoriatic arthritis, refractory rheumatoid arthritis, chronic non-rheumatoid arthritis, osteoarthritis, osteoporosis/bone resorption, ankylosing spondylitis, Crohn's disease, atherosclerosis, ulcerative colitis or psoriasis.

23-28. (canceled)
